(19) United States
(12) Patent Application Publication
(10) Pub. No.: US 2026/0232738 A1
(43) Pub. Date: Aug. 13, 2026

(54) ANAEROBIC ONCOLYTIC BACTERIAL FORMULATION

(71) Applicant: Northwestern University, Evanston, IL (US)

(72) Inventors: Dong Hyun Kim, Evanston, IL (US); Hyunjun Choi, Evanston, IL (US); Bongseo Choi, Evanston, IL (US)

(21) Appl. No.: 19/153,554

(22) PCT Filed: Feb. 8, 2024
(86) PCT No.: PCT/US2024/015032

§ 371 Date: Aug. 4, 2025

Related U.S. Application Data
(60) Provisional application No. 63/483,902, filed on Feb. 8, 2023.

(51) Int. Cl. A61K 35/742; A61K 9/00; A61K 9/48; A61K 9/50; A61K 35/00; A61P 35/00

(52) U.S. Cl. CPC A61K 35/742; A61K 9/0019; A61K 9/4858; A61K 9/5036; A61P 35/00; A61K 2035/115

(57) ABSTRACT

Disclosed are formulations and compositions of anaerobic oncolytic bacterium spores and calcium alginate microspheres, methods of using the formulations and compositions, such as for targeted delivery to a tumor and treatment of associated diseases and disorders.

### CROSS-REFERENCE TO RELATED APPLICATIONS

This application claims benefit of U.S. Provisional Application Ser. No. 63/483,902, filed Feb. 8, 2023, the entire contents of which is incorporated by reference herein.

### STATEMENT REGARDING FEDERALLY SPONSORED RESEARCH OR DEVELOPMENT

This invention was made with government support under grant numbers CA218659 and EB026207 awarded by the National Institutes of Health. The government has certain rights in the invention.

### BACKGROUND

The role of bacteria as an anti-cancer agent was recognized more than a century ago following observation that infections with bacteria improved outcomes in cancer patients. Various bacterial species have been found to have oncolytic activity, such as Salmonella, Listeria, and Clostridium. Recently, Clostridium novyi (C. novyi) was discovered to have the ability to germinate and populate in local tumors. C. novyi was rendered non-pathogenic by selecting a clone without the major toxin (α-toxin), named C. novyi-NT (NT: Non-toxic). It is known to be a promising bacteria for cancer therapy, that can be an effective tumor specific treatment and easily handled with their environmental resistant spore form. C. novyi-NT is an obligate anaerobe and a highly motile bacterium that is exquisitely sensitive to oxygen; vegetative forms cannot survive in oxygen while bacterial spores can only germinate in hypoxic conditions. C. novyi-NT spores germinate when exposed to selective hypoxic/necrotic tissue regions and destroy tumor cells through secretion of lipases, proteases, and degradative enzymes. Studies using systemic intravenous (IV) or intra-tumoral (IT) injection of C. novyi-NT spores in mice and rabbits have demonstrated remarkable efficacy, including often complete regression. The efficacy and relative safety have recently been demonstrated in companion canines with naturally occurring sarcomas; human clinical trials have been completed in patients with advanced solid tumors. Both animal model and initial clinical studies have demonstrated the potential of this developing bacteriolytic paradigm. However, several critical gaps-in-knowledge must be addressed to maximize the safety and efficacy of these methods, particularly for the treatment of patients with various solid tumors. Long-term toxicity has been demonstrated with systemic injection of spores.

Transcatheter intra-arterial infusion is now routinely used by interventional radiologists for image-guided intra-hepatic delivery of embolic materials, chemotherapeutics (TACE) and brachytherapy-beads. These catheter-directed therapies rely upon differences in the blood supply between liver tumors (primary or metastatic) and normal liver tissues. Limitations of percutaneous IT injection approaches include accessibility to colorectal cancer liver metastases (CRLM) that is dependent upon intra-hepatic position, and the number of concurrently treatable tumors is limited to the number of needle punctures that can be tolerated. Also, bacteriolytic studies have demonstrated that anaerobes administered via systemic or IT delivery routes can fail to treat completely normoxic tumor rim tissues.

Alternative approaches are needed to augment the delivery of oncolytic bacteria to targeted tumor or metastases, and to improve the safety and efficacy of bacterial oncolysis methods.

### SUMMARY

Disclosed are formulations and compositions of anaerobic oncolytic bacterium spores and calcium alginate microspheres, methods of using the formulations and compositions, including for targeted delivery, such as to a tumor, and treating diseases and disorders, such as diseases and disorders associated with a tumor. One aspect of the disclosure is an anaerobic oncolytic bacterial formulation or composition comprising anaerobic oncolytic bacterium spores and calcium alginate microspheres. In embodiments, the anaerobic oncolytic bacterium spores can germinate in the calcium alginate microspheres.

Another aspect of the disclosure is a method of delivering anaerobic oncolytic bacteria to a tumor in a subject by delivering or administering a formulation or composition containing anaerobic oncolytic bacterium spores and calcium alginate microspheres. In embodiments, the formulation or composition is delivered or administered by embolization, such as trans-arterial embolization. In the disclosed methods, the anaerobic oncolytic bacterium spores can germinate in the calcium alginate microspheres to vegetative anaerobic oncolytic bacteria. In further embodiments, microspheres containing vegetative anaerobic oncolytic bacteria release the vegetative anaerobic oncolytic bacteria to the tumor.

Further aspects of the disclosure are methods of treating a tumor in a subject comprising delivering or administering a formulation or composition containing anaerobic oncolytic bacterium spores and calcium alginate microspheres to the tumor. In embodiments, the formulation or composition is delivered or administered by embolization, such as trans-arterial embolization. In embodiments, the anaerobic oncolytic bacterium spores germinate in the calcium alginate microspheres to vegetative anaerobic oncolytic bacteria. In embodiments, the microspheres release the vegetative anaerobic oncolytic bacteria to the tumor. In the disclosed methods, the vegetative anaerobic oncolytic bacteria induce an anti-tumor response and/or induce an immune response against the tumor. Examples of inducing an anti-tumor response include inhibiting tumor cell growth, suppressing tumor cell growth, causing tumor cell death, and combinations thereof. Examples of inducing an immune response against the tumor include enhancing or increasing dendritic cells in the tumor environment.

In the embodiments, the anaerobic bacterium is a species of Clostridium, Listeria, or Salmonella. In embodiments, the anaerobic bacterium spores are non-toxic, such as C. novyi-NT.

Additional features and advantages of the formulations, compositions, and methods are set forth in the description below, and will be apparent from the description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

It should be understood that the drawings described below are not intended to limit the scope of the present disclosure in any way.

FIG. 1. is a schematic image of germination factors of Clostridium species.

FIG. 2. is schematic images showing of the mechanism of conventional C. novyi-based cancer therapy.

FIG. 3. is a schematic image showing potential effect of dipicolinic acid released from the germination of C. novyi spores in calcium alginate microspheres.

FIG. 4. is schematic images showing potential effect of endovascular intraarterial approach on the distribution of C. novyi in the tumor region.

FIG. 5. is optical microscopic images of Clostridium novyi spore-loaded calcium alginate microspheres (CS-CAMS).

FIG. 6. illustrates the quantification of vegetative C. novyi after the spore-loaded microsphere suspensions were incubated in hypoxia or normoxia for predetermined time points.

FIG. 7. is optical microscopic images of spore-loaded calcium alginate microspheres showing morphological difference by the incubation condition (hypoxia or normoxia). The optical microscopic images at bottom showed iron oxide-decorated spores for better visualization of spore germination activity.

FIG. 8. illustrates calcium quantification in calcium-added PBS ([Ca²⁺]=1 mM) after the initial germination of C. novyi spores incubated in hypoxic condition. (X-axis indicates the number of spores in the same volume of PBS).

FIG. 9. illustrates loss of OD assay for demonstration of calcium dependent enhancement of C. novyi.

FIG. 10. illustrates quantification of dipicolinic acid (DPA) released from the C. novyi spores during germination process.

FIG. 11. is optical microscopic images of CS-CAMS and C. novyi spore loaded barium alginate microspheres (CS-BAMS) before and after treatment of dipicolinic acid (DPA).

FIG. 12. illustrates comparative incubation test of calcium alginate and barium alginate for demonstration of calcium-dependent disruption of alginate microspheres.

FIG. 13. shows MR scan images of animal groups with hepatocellular carcinoma N1S1 tumor lesions treated with PBS (control), C. novyi spore, blank calcium alginate microspheres (CAMS), and CS-CAMS. (All agents were introduced intraarterially into proper hepatic artery using a microcatheter).

FIG. 14. illustrates tumor effect with hepatocellular carcinoma N1S1 following intraarterial administration of PBS, C. novyi spores, CAMS, and CS-CAMS after 14 days of treatment, by volume measurement.

FIGS. 15A-15B. is histological sections with gram staining of hepatocellular carcinoma (N1S1) cells: treated with PBS, C. novyi, and CS-CAMSs after 14 days post treatment (FIG. 15A); and treated with CS-CAMSs after 5 days post treatment (FIG. 15B).

FIGS. 16A-B. illustrate flow cytometry analysis of CD86+/CD80+ dendritic cells in groups treated with PBS, C. novyi, CAMS, and CS-CAMS; contour plot (FIG. 16A) and bar graph at 5-day and 14-day post-treatment (FIG. 16B).

FIGS. 17A-B. illustrate flow cytometry analysis of T lymphocytes in groups treated with PBS, C. novyi, CAMS, and CS-CAMS; contour plot (FIG. 17A) and bar graph at 5-day and 14-day post-treatment (FIG. 17B

FIGS. 18A-B. illustrate flow cytometry analysis of CD25−/FoxP3+ cells in groups treated with PBS, C. novyi, CAMS, and CS-CAMS; contour plot (FIG. 18A) and bar graph at 5-day and 14-day post-treatment (FIG. 18B).

FIG. 19. illustrates the effect of treatment with PBS, C. novyi, CAMS, and CS-CAMS on regulatory T cell population in CD4+ T lymphocyte.

FIG. 20. illustrates flow cytometry analysis of T-helper 17 cells in groups treated with PBS, C. novyi, and CS-CAMS.

### DETAILED DESCRIPTION

Disclosed are formulations, compositions, and methods for targeted delivery of anaerobic oncolytic bacteria. The delivery of anaerobic oncolytic bacteria can provide anti-tumor and immunomodulation activity to a tumor and associated diseases and conditions. Unless defined otherwise, all technical and scientific terms used in this disclosure with the appended claims have the same meaning that is commonly understood by one of ordinary skill in art to which the subject matter pertains. As used in the specification and the appended claims, unless specified to the contrary, the following terms have the meaning indicated to facilitate the understanding of the disclosure.

Unless otherwise specified or indicated by context, the terms “a”, “an”, and “the” mean “one or more.” In addition, singular nouns should be interpreted to mean “one or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

Also as used herein, “and/or” refers to and encompasses any and all possible combinations of one or more of the associated listed items, as well as the lack of combinations when interpreted in the alternative (“or”).

As used herein, the terms “include,” “including,” “contain”, “containing,” or “characterized by” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The terms “formulation” and “composition” may be used interchangeably throughout the disclosure. The terms “bacterium” and “bacteria” may be used interchangeably.

As used herein, “tumor” may include primary tumor and/or metastases.

Broadly, the disclosure is directed to anaerobic oncolytic bacterial formulations or compositions that have anti-tumor and immunomodulation activities. Oncolytic bacteria are a current option for treatment of tumors and metastases that can have an advantage over other, conventional treatment modalities by directly suppressing or destroying a tumor or tumor cell, and/or by stimulating an immune response against the tumor or tumor cell. There are many cytotoxic drugs in the market for eradication of cancers⁹. However, the non-targeted normal tissue toxicity that seriously affects the patients' vitality have been an obstacle in cancer treatment using cytotoxic agents². To overcome this clinical challenge, the trend in cancer therapies has been moving towards molecularly targeted therapy¹⁰ and immunotherapy¹¹. Firstly, the clinicians and scientists in the medical industry may have an agreement to the targeted treatment of cancer cell by distinguishing special markers or environment made of tumor tissues from normal ones¹⁰. Furthermore, not only targeting the special biology of tumor microenvironment, but immunotherapy also can actively pinpoint the tumor tissue to be visible and recognizable by immune system of patients by the immune modulation of cancer tissue that may maintain immunosuppressive tumor microenvironment using immune-checkpoint blockade such as anti-CTLA4, anti-PD-1, anti-PD-L1 and so on¹¹⁻¹³.

With regard to the current trend in cancer therapies, bacteria-based cancer therapies having selective target has a huge potential to be an effective alternative to conventional therapies. Typically, therapeutic bacteria strains have been chosen strictly for selective proliferation and therapeutic action in tumor regions². Furthermore, intrinsic immunogenic nature of bacteria can modulate the immunosuppressive tumor region to be proinflammatory significantly¹⁴.

For example, Salmonella has a strong preference to the hypoxic tumor tissue, attracting Salmonella to a tumor region effectively¹⁵. Not only expression of cytotoxic enzymes and toxins, Salmonella express natural immune adjuvants such as lipopolysaccharides¹⁶ and flagellin¹⁷, which can trigger the activation of toll-like receptor 4 and 5, respectively.

Plus, Listeria can preferentially grow in specific cell types such as antigen-presenting cells (APC). Especially in tumor, many APCs such as myeloid derived suppressor cells (MDSCs) are accumulated and inactivated, hindering proper immune response to fight against tumor cells. Listeria can selectively kill those immunosuppressive cells and this event causes reduction in the expression of anti-inflammatory cytokines, and it results in the immune activation of tumor microenvironment¹⁸.

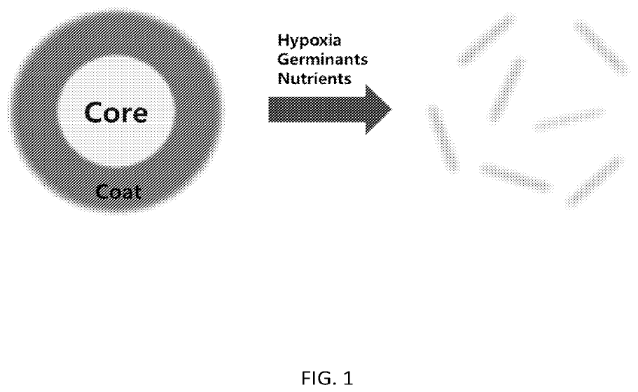

Species in Clostridia genus are gram-positive, anaerobic, endospore-forming bacteria¹⁹, and are one of the most popular bacteria adopted for cancer therapy². Clostridia is considered superior to other anti-cancer bacteria strains in terms of targeting tumor and safety because Clostridia is an obligate anaerobe that can survive only in the hypoxic tumor region and is an endospore forming bacterial genus, which allows rapid clearance of bioinert endospores from the blood and tissue effectively⁴. These bacteria initiate the sporulation process when they are under an unfavorable environment to survive, such as oxygen-rich conditions. The dormant endospores can sense the presence of oxygen, germinants and nutrients. When the environment around a spore becomes favorable for the survival of bacteria, spores can start the germination process for producing vegetative bacteria (FIG. 1)²⁰.

Disclosed are formulations and compositions of anaerobic oncolytic bacterium spores and calcium alginate microspheres, methods of using the formulations and compositions, including for targeted delivery, such as to a tumor, and for treating diseases and disorders, such as diseases and disorders associated with a tumor. Targeted delivery to a tumor may be directly to the tumor, to or near the tumor, or to the tumor region or environment.

One aspect of the disclosure is an anaerobic oncolytic bacterial formulation or composition comprising anaerobic oncolytic bacterium spores and calcium alginate microspheres. microspheres. The oncolytic bacterium spores may be in or within the calcium alginate microspheres, or contained or incorporated in the calcium alginate microspheres. In embodiments, the oncolytic bacterium spores are within or contained or incorporated in the calcium alginate microspheres.

In embodiments, the anaerobic oncolytic bacterium spores are capable of germinating in the calcium alginate microspheres. In some embodiments, the anaerobic oncolytic bacterium spores can germinate in the calcium alginate microspheres to vegetative anaerobic oncolytic bacteria. In some embodiments, the anaerobic oncolytic bacterium spores germinate to vegetative anaerobic oncolytic bacteria in the calcium alginate microspheres. In further embodiments, the microspheres containing vegetative anaerobic oncolytic bacteria can release the vegetative anaerobic oncolytic bacteria to the environment, such as to a tumor, or near the tumor, or to the tumor region or environment.

The disclosed anaerobic oncolytic bacterial formulation or composition includes anaerobic bacterium spores and calcium alginate microspheres. The anaerobic bacterium spores may be spores of any anaerobic bacterium that has oncolytic activity. Preferably, the anaerobic oncolytic bacterial strains show selective proliferation and therapeutic action in tumor or tumor regions. Also, the anaerobic oncolytic bacterial strains preferably may stimulate an immune response in a tumor or toward the tumor environment. In embodiments, the anaerobic oncolytic bacterium is selected from species of Clostridium, Listeria, and Salmonella. In some embodiments, the anaerobic oncolytic bacterium is Clostridium novyi. Preferably, the anaerobic oncolytic bacterium or anaerobic bacterium spore is not toxic.

By recombinant DNA modification, a Clostridium novyi clone that does not express α-toxin was developed for anti-cancer therapy²¹. Clostridium novyi-NT (C. novyi-NT; non-toxic C. novyi), an attenuated Clostridium novyi strain with deletion of lethal exotoxin genes, has been extensively studied in preclinical research and clinical trials for the clinical translation^{3, 21}. C. novyi-NT can express phospholipases, haemolysins, and lipases to kill tumor cells²², and also stimulate a potent immune response against the tumor²³. The C. novyi-NT spores do not cause infection or bacteria-related cytotoxicity and are quickly cleared from the body⁴. In embodiments of the disclosed compositions and methods, the anaerobic oncolytic bacterium is nontoxic. In some embodiments, the nontoxic oncolytic bacterium is Clostridium novyi, and the anaerobic bacterial spores in the compositions are C. novyi-NT spores.

In the disclosed embodiments, the anaerobic bacterium spores are in, within, or contained or incorporated in calcium alginate microspheres, for example suspended or encapsulated in the microspheres. Calcium alginate microspheres are known to be biocompatible carriers for biological substances and cells. In some embodiments, the anaerobic bacterium spores are C. novyi-NT spores. In some embodiments, C. novyi-NT spores are within, or contained or incorporated in calcium alginate microspheres (CS-CAMSs). It would be understood that the size of the microspheres may vary depending on the usage of the formulation or composition. For example, the microspheres may be used as a carrier for delivery by intra-arterial injection, such as embolization, or transcatheter intra-arterial injection, or infusion, particularly through tumoral vessels such as hepatic artery or tumor feeding vessels. A size range of the microspheres for such intra-arterial injection may be from about 0.1 um to about 500 um. The size range may be about 1 um to about 500 um, about 10 um to about 500 um, about 20 um to about 500 um, about 20 um to about 200 um, about 1 um to about 100 um, about 10 um to about 100 um, about 25 um to about 100 um, or about 25 um to about 75 um. Preferably, the size is a mean of about 50 um, 55 um, 60 um, 65 um, 70 um, 75 um, 80 um, 85 um, 90 um, 95 um, or 100 um. Also, the microspheres may be used as a carrier for delivery by intratumoral or percutaneous injection. A size range of the microspheres for such intratumoral or percutaneous delivery may be from about 0.1 um to targeted solid tumor size of about 5 cm. The size range may be about 1 um to about 5 cm, about 10 um to about 5 cm, about 20 um to about 5 cm, about 50 um to about 5 cm, about 75 um to about 5 cm, about 100 um to about 5 cm, about 1 um to about 1 cm, about 10 um to about 1 cm, or about 50 um to about 1 cm, about 100 um to about 1 cm, or about 200 um to about 1 cm. Preferably, the size range may be about 20 um to about 500 um, about 20 um to about 250 um, about 50 um to about 250 um, about 75 um to about 250 um, about 20 um to about 100 um, about 50 um to about 100 um, or about 75 um to about 100 um.

In the disclosed embodiments, the anaerobic bacterium spores in the calcium alginate microspheres (e.g. CS-CAMSs) may be synthesized by methods such as spraying-coagulation method. In embodiments, the anaerobic bacterium spores may be loaded within or incorporated in the calcium alginate microspheres in a range of 1×10\hat{ }3 to 3×10\hat{ }8 spores per ml, or about 1×10\hat{ }3, 1×10\hat{ }4, 1×10\hat{ }5, 1×10\hat{ }6, 1×10\hat{ }7, or 1×10\hat{ }8 per ml. In some embodiments, the anaerobic bacterium spores in the calcium alginate microspheres are about 1×10\hat{ }8 spores per ml.

The disclosed formulations or compositions and methods include or use anaerobic oncolytic bacterium spores that can germinate in the calcium alginate microspheres. In embodiments, the anaerobic bacterium spores germinate in the microspheres under hypoxic conditions. In embodiments, the anaerobic bacterium spores do not germinate in the microspheres under normal oxygen conditions.

### Methods of Use

In another aspect, the disclosure is directed to uses of the disclosed anaerobic oncolytic bacterial formulation or composition. Embodiments include use of the anaerobic oncolytic bacterial formulation or composition to deliver anaerobic oncolytic bacteria or anaerobic bacterium spores, such as to or near the tumor, or to the tumor region or environment, or to inhibit growth of a tumor. An embodiment is a method of delivering anaerobic oncolytic bacteria or anaerobic bacterium spores to or near the tumor, or to the tumor region or environment. Another embodiment is a method of delivering anaerobic oncolytic bacteria to a tumor in a subject comprising delivering an anaerobic oncolytic bacterial formulation or composition having anaerobic bacterium spores and calcium alginate microspheres. In the embodiments, the formulation or composition may be delivered directly to or near the tumor, or to the tumor region or environment. In embodiments, the tumor may be any tumor, malignant or benign. In some embodiments, the tumor is a solid tumor, including primary and metastatic tumors. As used herein, tumor may include primary tumor and/or metastases. Thus, in the methods related to deliver of anaerobic oncolytic bacteria or anaerobic bacterium spores to a tumor, the tumor may include primary tumor and/or metastases.

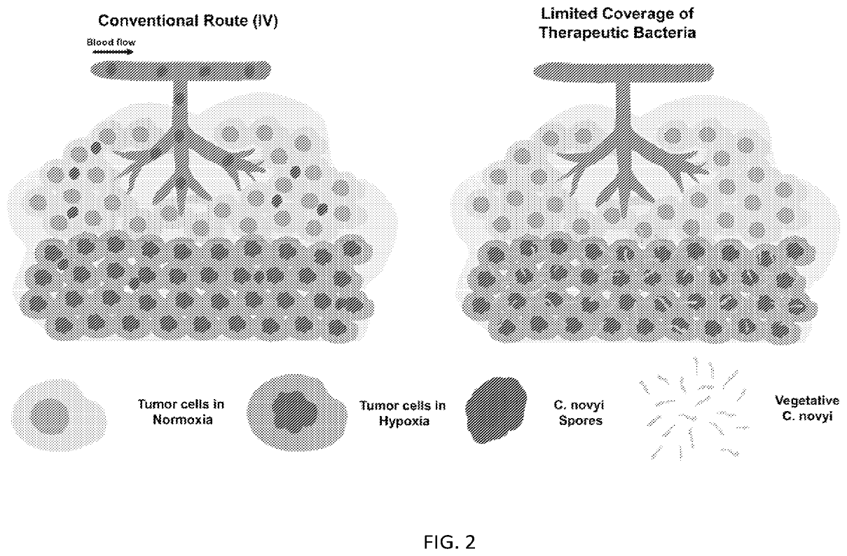

A solid tumor may be composed of proliferative tumor rim that has a duct of blood vessel and hypoxic necrotic tumor core (FIG. 2). Spores of C. novyi can germinate to vegetative C. novyi in the hypoxic necrotic tumor regions selectively and kill tumor cells. Air-dried C. novyi spores can be administrated by IV or IT injection directly²⁴. However, IV injection of spores showed limited therapeutic efficacy due to their poor biodistribution⁴, and spores injected intratumorally tended to germinate and kill tumor in necrotic regions selectively instead of the proliferative regions²⁵. This biological barrier for C. novyi to survive often leads to progression or relapse of the tumor^{7, 8}. Thus, localizing C. novyi spore precisely to near the tumor region and providing sufficient hypoxia covering proliferative tumor regions for C. novyi spores to germinate efficiently may bring significant enhancement of therapeutic efficacy of using these oncolytic bacteria. In embodiments of the methods of delivering anaerobic oncolytic bacteria to a tumor, the compositions are localized to a target region of the tumor, near the tumor or region, or to the tumor directly.

In endovascular interventional therapy for cancer treatment, embolization therapy is one of the mostly adopted strategies, and the embolization causes severe hypoxia on the downstream of occluded blood vessels²⁶. There are many hints that embolization can trigger the proliferation of anaerobic bacteria and infection of tissues, supported by cases reporting the sepsis by bacterial infection after the embolization procedures to treat hepatocellular carcinoma and uterine fibroid^{27, 28}. These case reports were about the side effect of embolization therapy but provide clues for potential combination of embolization and anaerobic bacteria cancer therapy.

In the disclosed methods, the formulation, or composition may be delivered by embolization. In particular embodiments, the embolization is transcatheter arterial embolization or trans-arterial embolization. In the embodiments, the embolization induces a hypoxic environment in the tumor, preferably throughout the tumor. In the example of a solid tumor, hypoxia is induced in the tumor rim or periphery that interacts with blood vessel that allow for oxygen through the tumor core (e.g., necrotic core).

In the disclosed methods, the anaerobic bacterium spores germinate in the microspheres. As a delivering carrier of C. novyi spores, the microsphere material should be able to perform as an embolic agent for inducing hypoxia to downstream target tissue. Also, the carrier material should provide essential germinants and nutrients to grow vegetative C. novyi efficiently because hypoxia is not a sole factor for germination and proliferation of Clostridia²⁹. For that, hydrogels are one of the suitable materials, enabling the exchange of molecule between environment and gel matrix by highly swellable nature³⁰. Plus, the material should be able to release vegetative C. novyi efficiently to enable the bacterial activity to reach tumor regions. Therefore, it is crucial to select the hydrogels that can be degradable by bacterial activities. Germinants are molecules that can help trigger the germination of bacterial spores. Most of germinants can induce the degradation of spore coat molecules and the hydration of spore core for turning on the gene activity of bacteria²⁰. Germination of bacterial endospores triggered with calcium ions and chelating agents was reported previously³⁶. Also, calcium ion are known as a co-germinant in C. difficile that is in Clostridia genus, but there is no information that calcium is a co-germinant in C. novyi²⁹. In embodiments, the microspheres are calcium alginate hydrogel microspheres, and the C. novyi spores use calcium ions from the calcium alginate microspheres for germination.

In some embodiments of the disclosed methods, the anaerobic bacterium spores are nontoxic C. novyi-NT spores that use calcium ions as germinants. In embodiments, C. novyi-NT spores use calcium ions in the microspheres for germination. The calcium ions may be used to initiate germination and/or sustain germination in the microspheres. In embodiments, the microspheres are calcium alginate hydrogel microspheres, and the nontoxic C. novyi-NT spores use calcium ions from the calcium alginate microspheres for germination.

In disclosed embodiments, the anaerobic oncolytic bacterium spores can germinate to vegetative anaerobic oncolytic bacteria in the calcium alginate microspheres. In embodiments, the microspheres contain vegetative anaerobic oncolytic bacteria.

In some embodiments, the C. novyi-NT spores use calcium ions in the microspheres along with co-germinants, such as additional calcium in the tumor environment. Other co-germinants may include amino acids, for example L-alanine, L-arginine, L-phenylalanine, L-cysteine, L-serine, L-valine, L-threonine, L-asparagine, L-glutamine, glycine, methionine, etc. Additional co-germinants may be organic molecules, such as deoxycholate, exogenous dipicolinic acid, L-lactate, pyruvate, sodium bicarbonate, etc.

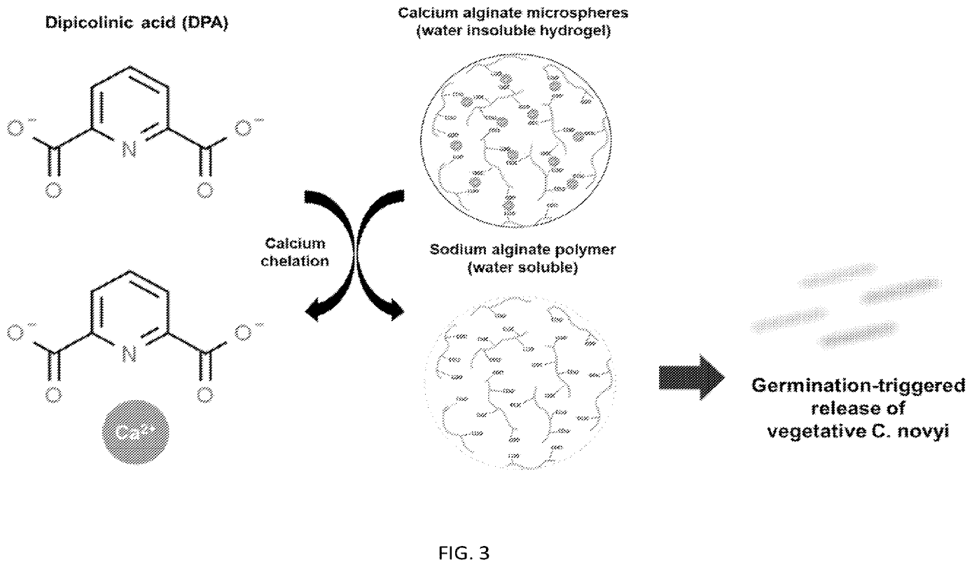

It is known that potential genes that may digest alginate are coded in C. novyi genome²². Also, calcium ions are one of the critical factors to trigger germination of Clostridia²⁹. In addition to the effect of calcium germinating many strains of Clostridia genus, the internally packed dipicolinic acid (DPA) in the core of the spore has a strong interaction with metal ions. Furthermore, DPA can be released out from the spore core in the early germination stage. These hints may indicate the bacterial germination activity may cause the chelation of calcium ions in the matrix of calcium alginate microspheres which may be able to disrupt the morphology of microsphere by swelling and/or dissolution of alginate polymer chains (FIG. 3). The disruption of calcium alginate microspheres triggered by the germination process of C. novyi further enhance the release of vegetative C. novyi to the tumor region. In the disclosed methods, C. novyi-NT spores contain dipicolinic acid. In embodiments, dipicolinic acid in the spore chelates calcium ions from the calcium alginate microspheres. The DPA chelating calcium ions in the calcium alginate microspheres degrade the internal crosslinks and the morphology of microspheres, which causes the microspheres to swell and degrade.

In the disclosed methods, microspheres containing anaerobic bacterium spores germinated to vegetative oncolytic bacteria are degraded and release the vegetative oncolytic bacteria into the tumor and tumor environment. In embodiments, the vegetative oncolytic bacteria induce anti-tumor response in the tumor. In embodiments, the vegetative oncolytic bacteria induce an immunomodulation effect against the tumor and tumor cells.

In another aspect, the disclosed anaerobic oncolytic bacterial composition may be used to treat a tumor in a subject. In embodiments, the tumor may be malignant or benign. In some embodiments, the method is directed to treating a subject having a cancer.

The methods may be directed to a method of treating a tumor in a subject involving delivering the anaerobic oncolytic bacterial composition described herein. As disclosed herein, method may involve delivering the anaerobic oncolytic bacterial composition by embolization that induces or expands a hypoxic environment in the tumor. As a result of the hypoxic condition and the calcium uptake by the spores during germination, the microspheres degrade and release vegetative anaerobic oncolytic bacteria in the tumor. The anaerobic oncolytic bacteria induce an anti-tumor and immune responses in the tumor.

In embodiments, the anti-tumor response includes inhibiting tumor cell growth, suppressing tumor cell growth, causing tumor cell apoptosis and tumor death, and combinations thereof. In the embodiments, the anaerobic oncolytic bacteria affect part of or the entire tumor region. In some embodiments, the anaerobic oncolytic bacteria affect the tumor rim or periphery through the vasculatures due to induced hypoxia.

In embodiments, the immune response enhances major immune cells related to tumor lysis. Dendritic cells in a tumor are the major antigen presenting cells modulating adaptive immune system by activating or deactivating T cells. In embodiments, the immune response enhances or increases dendritic cells in the tumor. Further, the immune response from the anaerobic oncolytic bacteria enhances the activity of Th17 cells and decreases the function of regulatory T cells.

The compositions may include pharmaceutical solutions comprising carriers, diluents, excipients, and surfactants as known in the art. Further, the compositions may include preservatives. The compositions also may include buffering agents.

The disclosed formulations, compositions, and methods may be used to deliver or administer the composition to a subject, or to treat disorders and diseases associated with a tumor in a subject. The terms “subject” or “patient” may be used interchangeably throughout the disclosure and may refer to all animals, including mammals, e.g., human or non-human.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

### EXAMPLES

Clostridium. novyi (C. novyi) is one of the therapeutic bacterial species for treatment of solid tumors^{1, 2}. They have been tested for the anticancer therapy of various solid tumors in clinical trials³. Unlike other bacterial strains, obligate anaerobe, C. novyi has many advantages for safe tumor treatment. C. novyi can selectively grow in hypoxic region of a tumor and kill tumor cells while many bacteria can show severe toxicity on normal tissue⁴. Also, C. novyi is endospore forming bacteria that can be easily stored in many formulations like other typical drugs, which can be a significant benefit for production and storage^{5, 6}. However, the critical challenge of C. novyi bacteria as an antitumor agent is their limited prosperous clinical translation of C. novyi-related therapy. C. novyi show severe cytotoxicity in hypoxic and necrotic core region in the tumor, while cancer cells in highly vascularized viable tumor rims still proliferate vigorously without hinderance by bacterial activities. The proliferation of cancer cells in tumor rims causes a high chance of recurrence of tumor after treatment of C. novyi^{7, 8}.

Therefore, to overcome this limitation, the adoption of induced hypoxia burrowing endovascular technology can be a key to cover all of the tumor region with hypoxia to enlarge the bacterial territory and to enhance the therapeutic response. Also, it is well-known that the trans-arterial delivery of therapeutic substances using endovascular technology can enhance the delivery efficiency of a drug. Utilizing endovascular therapy, the effective delivery of C. novyi spores may be enabled.

For proof, alginate-based embolic material was designed as a C. novyi spore loading vehicle and tested for demonstrating the mechanism of action in this system. Also, the effect of embolization on the tumor eradication by C. novyi activity was explored. Furthermore, the immune modulation of tumor by combinational treatment of C. novyi therapy and embolization was determined.

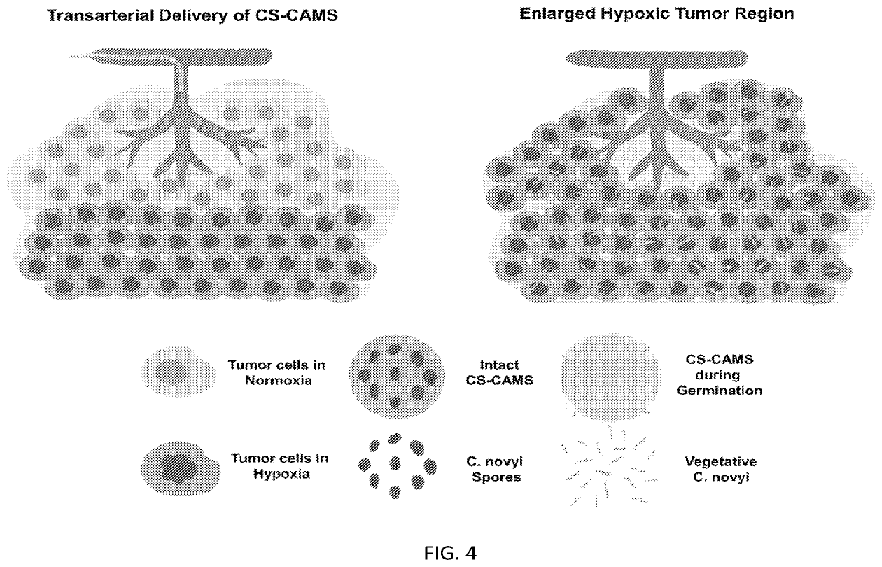

C. novyi spores loaded in calcium alginate microspheres (CS-CAMSs) were monitored by an optical microscope to track the germination process in alginate hydrogel when CS-CAMS are incubated under hypoxic conditions. Also, degradation mechanisms of CAMSs by bacterial activities were demonstrated. Furthermore, transcatheter intraarterial injection of CAMS to rat hepatic artery were performed to determine the expansion of territory for C. novyi activity in tumor with in vivo study and to compare therapeutic advantages using specialized formulation for intraarterial delivery compared with conventional delivery of C. novyi spores (FIG. 4).

Here, a multifunctional delivery vehicle of Clostridium novyi (C. novyi) spores for enhancing the dosage to tumor and the distribution of therapeutic bacteria in the tumor was developed. C. novyi spores were successfully immobilized in a matrix of calcium alginate microspheres and could be germinated to vegetative C. novyi bacteria effectively in the microspheres. Furthermore, by swelling and degradation of calcium alginate microspheres, the living C. novyi bacteria could escape from the microspheres and migrate to the media.

The possible mechanism of swelling and degradation phenomenon of C. novyi spore loaded alginate microspheres was explored. The calcium ions forming the crosslinking between alginate polymer chains could be captured competitively by germination process. The early germination stage effectively captured the calcium ions in media, and it was found out that C. novyi spores utilized the calcium ion in media as a co-germinant. Also, dipicolinic acid (DPA) that is a molecule highly accumulated in the core of C. novyi spore could effectively chelate the calcium ions in the matrix of alginate microspheres and degrade the microsphere formation. With a plenty of evidence, calcium alginate microspheres showed the strong potential as a delivery vehicle for C. novyi spores.

In animal studies, the combinational therapy of embolization and bacterial immunotherapy using CS-CAMS showed strong enhancement of anti-cancer response in a rat hepatocellular carcinoma model. Also, the histology proved the hypothesis that delivery of C. novyi spores using embolic agent could effectively delivered the C. novyi and expand the area of viable C. novyi that could perform cytotoxicity on tumor tissue covering vasculature-rich tumor rim.

In investigation for immune modulation by intraarterial treatment of CS-CAMS, CS-CAMS treatment induced superior activation of dendritic cells that are responsible for activating adaptive immune responses. Also, the treatment of CS-CAMS triggered CD4+ T cell mediated immune responses against tumor, showing reduced FoxP3+ regulatory T cells. Furthermore, the possibility of involvement of T-helper 17 cells in this therapeutic scenario was explored.

The system may contribute to overcoming current challenges of C. novyi bacteria-based therapies by adoption of endovascular technology and temporary embolization providing smart control of bacterial germination site and the area of therapeutic bacteria can thrive for suppressing or killing tumor cells.

### Methods

### Materials

Non toxin (NT) strain of Clostridium novyi (C. novyi-NT) was kindly provided by BioMed Valley Discoveries (Kansas City, MO, USA). Sodium alginate (MW 10,111), calcium chloride (CaCl₂), barium chloride (BaCl₂) and dipicolinic acid (DPA) were purchased from Sigma-Aldrich (St Louis, MO, USA). C. novyi culture media was prepared with 5 g Na2HPO4, 30 g peptone, 0.5 g L-cysteine, 10 g maltose, and 5 w/v % dried cooked meat particles (Difco) in 1 L of distilled water. Calcium chloride and sodium alginate were dissolved into Milli-Q grade water and filtered for further use. Other reagents and materials were used for further experiments without purification.

### Synthesis of C. novyi Spore Loaded Alginate Microspheres

C. novyi spore loaded calcium alginate microspheres (CS-CAMSs) were synthesized with spraying-coagulation method.³⁵ Briefly, 6 mL of 3 w/v % sodium alginate solution was mixed with 3 ml of C. novyi spore suspension containing 3×10⁸ spores (Final alginate concentration: 2 w/v % and final concentration of C. novyi spore (1×10⁸ spores/mL).

The mixture was sprayed with high-volume low-pressure (HVLP) air-assisted spray gun (10 bar) on to the reservoir containing 0.5 M CaCl₂. The synthesized microspheres in CaCl₂ reservoir were further solidified for additional 1 hour. Then, CS-CAMSs were washed with Milli-Q grade water for 3 times for further use. The synthesized microspheres may be refrigerated or freeze-dried for storage.

### Characterization of CS-CAMSs

CS-CAMSs were observed with optical microscope (Olympus, Japan), and the 200 particles in the microscopic images were counted for calculation of mean size of particles. For the vegetative C. novyi release test, the multiple CS-CAMS (0.2 mg/mL) loaded culture dishes was incubated in hypoxic chamber (BD Gaspak™ MEZ Anaerobe Pouch System (Sparks, MD, USA)) or normoxic condition in 37° C. Next, the individual dishes were collected and frozen in −80° C. freezer. After the final collection of samples of 96-hour incubation, supernatant solutions of all samples were seeded in 96 well plate for the measurement of the concentration of C. novyi escaped or released from CS-CAMSs. The concentration of bacteria was measured with typical OD 600 method using UV-Vis spectrometer.

### Mechanism Studies for CS-CAMS Degradation

The morphology change of CS-CAMSs was tracked with optical microscopic imaging during the incubation of 0.2 mg/mL of CS-CAMS in C. novyi medium or Dulbecco's modified eagle's medium (DMEM) in hypoxic or normoxic condition. For the visualization of C. novyi spores immobilized in calcium alginate microspheres, iron oxide nanoparticles (Feraheme®, Amag Pharmaceuticals, MA, USA) were decorated with spores.

### Effect of Calcium Ions on Germination of C. novyi

Calcium chloride salt was added to calcium-free phosphate buffered saline (PBS) for the preparation PBS containing 1 mM Ca²⁺. Next, calcium concentration in PBS before and after triggering germination of C. novyi was measured with the colorimetric calcium quantification assay kit using UV-vis spectroscopy and the fluorescent quantification assay kit using fluorometer. To identify the effect of calcium ions on germination of C. novyi spore, the calcium and glycine (mM) containing PBS was prepared with various calcium ion concentration (0-60 mM). The deoxycholate and glycine containing PBS was prepared as a positive control, which is known combination for triggering germination of Clostridium spores. 1×10⁶ of C. novyi spores were seeded in the media, and the germination was tested for up to 2 hours. OD 600 of the collected sample was measured to quantify the loss of OD 600 indicating the germination effect by spore core hydration³¹.

### Effect of Dipicolinic Acid on CS-CAMSs

The release of dipicolinic acid (DPA) from C. novyi spores was quantified with terbium DPA coordination methods³². Briefly, C. novyi spores were incubated in the C. novyi media under hypoxia for triggering germination process, and during the incubation, the samples were collected at predetermined time points and filtered with centrifugal filter (3000 D Molecular cut-off) for isolating small molecules from the media. Next, the samples were mixed with 1 mM terbium chloride solution, and the fluorescence intensity was measured with fluorescence spectroscopy (excitation 272 nm/emission 545 nm). To determine the chelating effect of the dipicolinic acid (DPA) on CS-CAMSs, calcium (CS-CAMS) or barium (CS-BAMS) alginate microspheres encapsulating C. novyi spores were fabricated with spraying coagulation method. For the synthesis of CS-BAMSs, 0.5 M BaCl₂ solution was used as a reservoir for solidification of microdroplets. CS-CAMSs and CS-BAMSs containing suspensions were treated with pH 7.4 DPA solution (Final concentration of DPA: 400 μM) to test the morphology of microspheres. Also, the morphology of CS-CAMS and CS-BAMS was compared in optical microscopy during incubation in C. novyi media under hypoxia for up to 24 hours.

### Tumor Challenge Study and Intra-Arterial Injection Procedure

All experiments were approved by our Institutional Animal Care and Use Committee (IACUC). N1S1 hepatoma cells derived from Sprague Dawley (SD) rats were cultured with Iscove's Modified Dulbecco's Medium (IMDM), and 3×10⁶ cells in 100 μl PBS were inoculated in the left lateral lobe of liver region of rats after the laparotomy. Incision closure for the muscle layer and the skin was performed using 4-0 absorbable Vetacryl™ sutures (Ethicon, Somerville, NJ, U.S.). After the surgery, to provide post-operative analgesic agent, Meloxicam (Loxicom, Norbrook, Newry, Northern Ireland, UK) at 2 mg/kg was administered by subcutaneous injection. After 7-days post tumor induction, the size of the tumor was monitored using Bruker 7.0 T ClinScan high-field small animal MRI (Bruker BioSpin, Ettlingen, Germany). After the tumor volume reached over 4 mm³, for intra-arterial delivery of PBS, C. novyi spores, and CS-CAMSs, the N1S1 bearing SD rats were gently anesthetized with isoflurane gas, and the incision was performed to abdomen, and the duodenum was searched and pulled down to reveal major bunch of portal triad (portal vein, bile duct, and hepatic artery). The connective tissue on bile duct branch connected to duodenum was thoroughly dissected to isolate gastroduodenal artery from the bile duct. Next, the distal side of the gastroduodenal artery was sutured with silk sutures to prevent the reflux, and the blood flow from common hepatic artery connected to aorta was blocked with a bulldog clamp. 24½G angiocatheter (SURFLASH, Terumo Medical Co, Somerset, NJ, U.S.) was inserted through the gastroduodenal artery and advanced to the proper hepatic artery. The small amount of heparin solution was injected with the catheter to prevent blood clot formation. Then, 0.5 mL of PBS was injected to check the successful catheterization without leakage, and the samples dispersed in 500 μL of PBS were slowly infused through the catheter. After the injection, the hole made by catheterization was treated with silk sutures, and the bulldog clamp on the common hepatic artery was gently removed. After checking the bleeding on blood vessel treated, the incision closure procedure was performed on muscle and skin layer. After the treatments, the tumor response was monitored using MRI up to 14-days post treatment. The distribution of C. novyi in the tumor was stained with gram staining. Crystal violet was used for staining proteoglycan of gram-positive bacteria, and Safranin was used for counter staining.

### Immune Characterization of CS-CAMS Treated Tumors

After sacrifice of N1S1 bearing SD rates treated with PBS, C. novyi spores, CAMS, and CS-CAMSs, the tumor were collected by the dissection of the liver lobe where the tumor lesion invaded after the perfusion of blood with saline solution through right ventricle of the heart. Collected tumor samples were mechanically homogenized and sieved to exclude the connective tissue and cell aggregations using cell strainers (40 μm, Corning, Corning, NY, U.S.). Red blood cells were lysed by using ACK lysis buffer (Thermo Fisher, Waltham, MA, U.S.) for 3 min at room temperature. Purified cells (1×10⁶ cells) in tumor were stained with indicated rat antibodies (CD11c, CD80, CD86, CD3, CD4, CD8, CD25, FoxP3, and IL-17 (BD Biosciences, Franklin Lakes, New Jersey, US)) to evaluate the population of immune cells in the tumor tissue. Specifically, the maturation of DC was analyzed from the double positive population of CD80+/CD86+ among CD11c+ cells. CD8+ T cells and CD4+ T cells were distinguished from CD3+ lymphocyte pool using CD4 and CD8 antibodies. Regulatory T cells (Treg) were selected with the double-positive population of CD3+/CD4+ T cells initially, and the population with double-positive of CD25−/FoxP3+ was counted. Also, T-helper 17 cells (Th17) were counted from the population with the expression of IL-17 among CD3+/CD4+ lymphocytes. Flow cytometry analysis for immune characterization of cells in treated tumor was performed using LSR Fortessa II (6 lasers, BD Biosciences, Franklin Lakes, NJ, U.S.)

### Example 1 Synthesis of C. novyi Spore-Loaded Alginate Microspheres

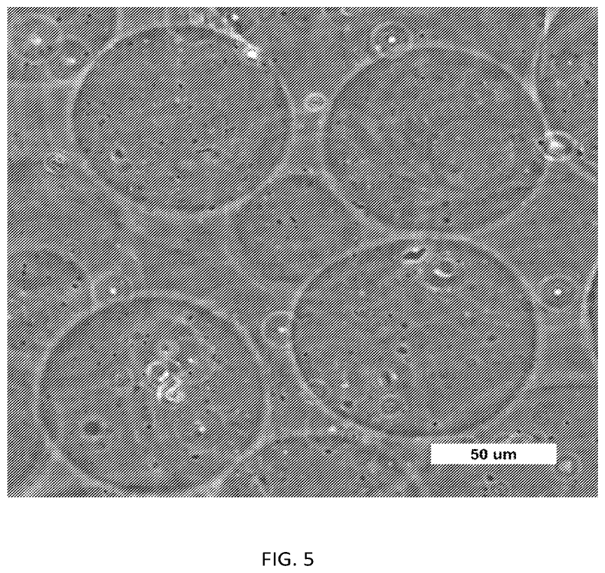

Calcium alginate microspheres have shown an advantage of encapsulating living organisms such as stem cells for tissue engineering^{33, 34}. The synthetic environment of calcium alginate microspheres using spraying-coagulation method is known to be ambient and biocompatible for loading biological substances and cells³⁵. As shown in FIG. 5, the calcium alginate microspheres encapsulating C. novyi spores (CS-CAMSs) were successfully synthesized with the spraying-coagulation method, and the mean size of microspheres was 75 μm, which is suitable for the embolic microspheres to occlude the hepatic arteries without causing pulmonary embolism that may be caused by small microparticles under the size of 10 μm. Also, the small dots indicated the incorporated spores in the matrix of microspheres.

### Example 2. Spore Germination Study of C. novyi Spores in CS-CAMS

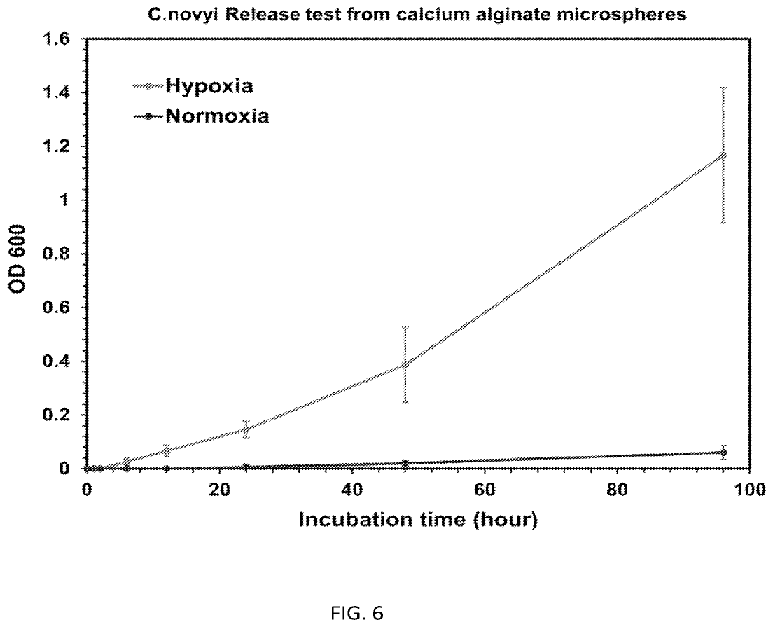

The ability of CS-CAMS to effectively encapsulate the C. novyi spores with minimal leakage of spore particles from the hydrogel was tested by comparing the two groups incubated in either hypoxic condition that provided less oxygen or normoxic condition with normal atmospheric oxygen level. After the incubation of predetermined time (0 to 96 hours), the supernatants were collected and re-incubated in hypoxia for additional 24 hours to quantify the number of spores and/or vegetative bacteria released out from the CS-CAMSs. FIG. 6 shows the absorbance of supernatant at 600 nm that is a typical method for quantifying bacteria in suspension. In this experiment, the OD600 would represent the amount of C. novyi spores and vegetative C. novyi released from the CS-CAMS during incubation. As shown in the navy plot indicating normoxia, the leakage of spores was strongly minimized in 37° C. On the other hand, the CS-CAMS incubated in hypoxic condition released significantly more amount of the vegetative C. novyi to the supernatant as shown in the red plot showing OD₆₀₀ over 1 in average (96 hours of incubation in hypoxia). These results implied the CS-CAMS can act in targeted hypoxic area in the body without activating the germination of C. novyi in normal tissue condition in the body, which may cause serious side effects in non-target area.

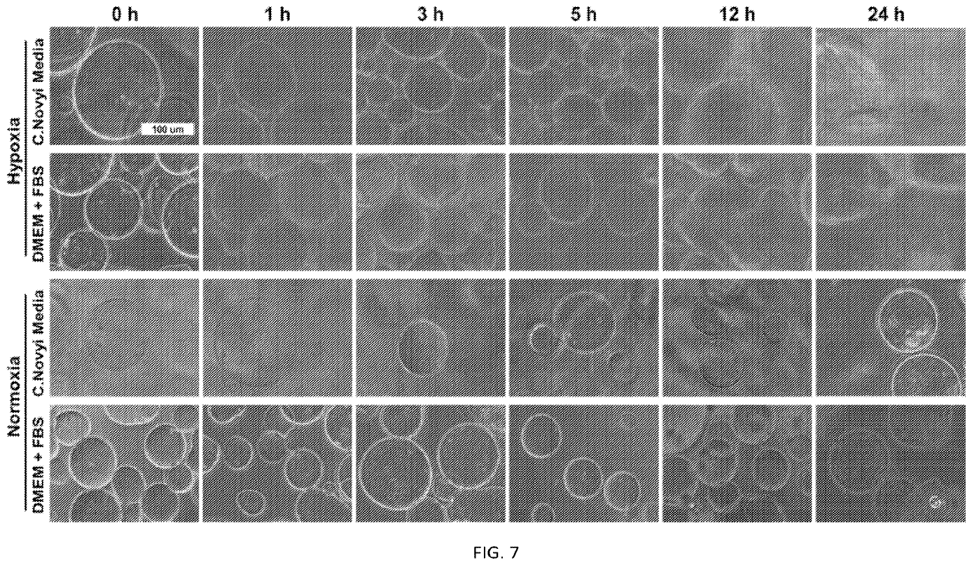

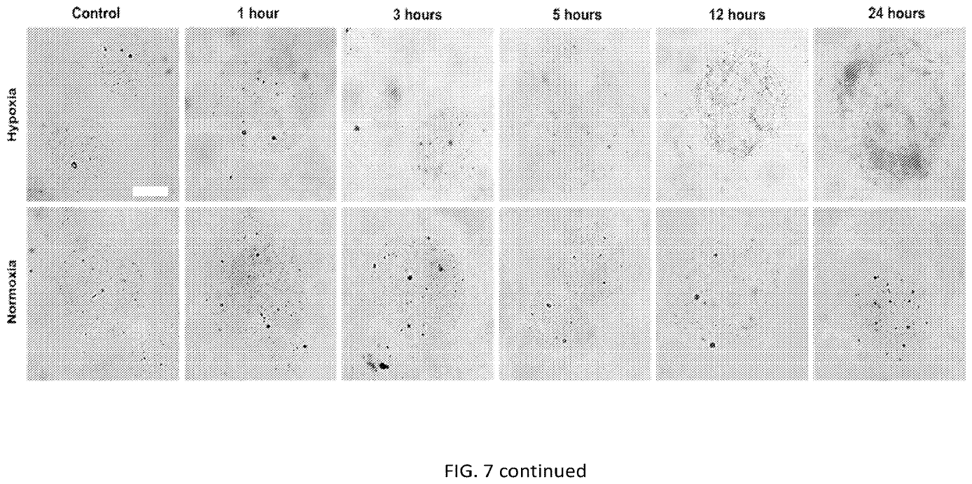

To determine the morphological change of CS-CAMS during bacterial germination, CS-CAMS samples were incubated in C. novyi media, which contains complete germinants and nutrition for spore germination and bacterial vegetative proliferation, or DMEM as a representative medium for mammalian cell culture. Two groups of CS-CAMSs were incubated for a predetermined time (1, 2, 3, 5, 12 hours) up to 24 hours and the morphology of microspheres was observed with the optical microscope. Surprisingly, after 12 hours of the incubation of CS-CAMSs in both C. novyi media and DMEM in anaerobic condition, the disintegration of CS-CAMSs was observed in microscopic images (FIG. 7). In contrast, CS-CAMS incubated in normoxic condition did not show the degradation of microspheres and showed small morphological defects during incubation due to temperature and salt effect. This phenomenon indicated the release mechanism of C. novyi bacteria was the triggered swelling effect of microsphere matrix caused by bacterial germination activity and/or vegetative bacterial activity.

### Example 3. Effect of Calcium Uptake of C. novyi Spores on CS-CAMS

To determine the mechanism of swelling of CS-CAMSs, two possible mechanisms induce the swelling of hydrogel. One mechanism can be calcium ions forming crosslinks between alginate polymer chains, and the chelating of calcium ions can trigger the swelling and disintegration of microspheres. The other mechanism can be enzymatic cleavage of glycosidic bonds linking among monomers. However, the genetic information about the alginase coding genes or homologous genes which are hydrolytic enzymes for cleavage of alginate chains were not found in previous publications dealing with genomic information of C. novyi²². Therefore, prior to elucidating the genetic expression of C. novyi, determining calcium ion-related metabolism of C. novyi germination was a rational and experimentally approachable study to understand the degradation of calcium alginate microspheres incorporated with C. novyi spores.

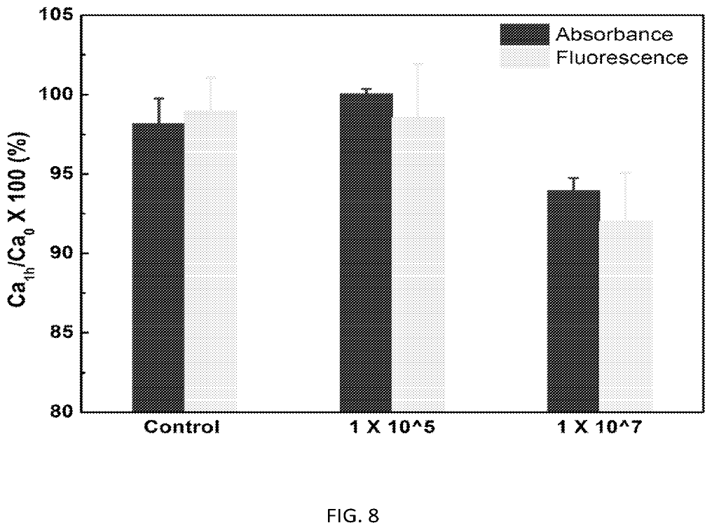

First, the calcium level during the germination process of C. novyi spores was measured using colorimetric and fluorescent quantification kits. As shown in FIG. 8, number dependent decrease of calcium level was observed in both colorimetric (from 98% to 93.9%) and fluorescent (from 99% to 92%) calcium quantification kit, indicating that C. novyi spores took calcium ions up in the process of germination after 1 hour of incubation in hypoxic condition. This initial interaction with calcium ion and spore can contribute the swelling of microspheres.

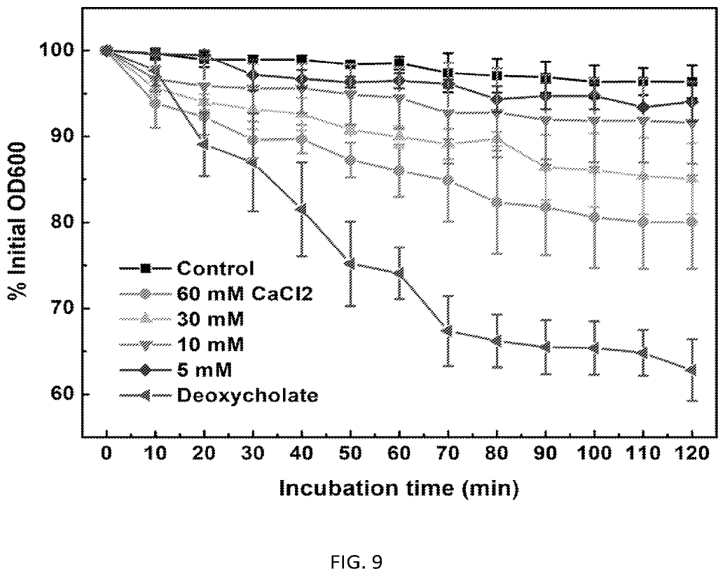

Germination of bacterial endospores triggered with calcium ions and chelating agents was reported previously³⁶. Also, calcium ion are known as a co-germinant in C. difficile that is in Clostridia genus, but there is no information that calcium is co-germinant in C. novyi²⁹. Clostridia genus shares similar genomic information among species^{37,38}, so the ability of calcium ions for inducing germination of C. novyi spores in the presence of glycine that is one of the essential co-germinants previously reported³⁹ was tested with loss of OD₆₀₀ assay. The loss of OD₆₀₀ assay determines the hydration of spore indicating actual start of germination process. OD₆₀₀ is absorbance of visible light at 600-nm wavelength, and the bacteria and spore particles reduce the amount of the light transmitted to the sensor. When the spores start germination, degradation of spore coat material and hydration of core induce the spore to be more transparent than the intact dormant spores. This phenomenon can be detected with OD₆₀₀ loss. As shown in FIG. 9, calcium ion thus could induce the loss of OD₆₀₀ compared to control group incubated in only glycine solution. Also, the loss effect was proportional to the concentration of calcium ions. Of course, the one of the mostly known germinant, deoxycholate³⁹ could induce more germination of C. novyi spores. These results indicated C. novyi spores utilize calcium ions for their germination, and environmental calcium ions can be a co-germinant for C. novyi. This biological activity of C. novyi spore during germination might contribute to the swelling of calcium alginate microspheres.

### Example 4 Effect of Dipicolinic Acid CS-CAMS

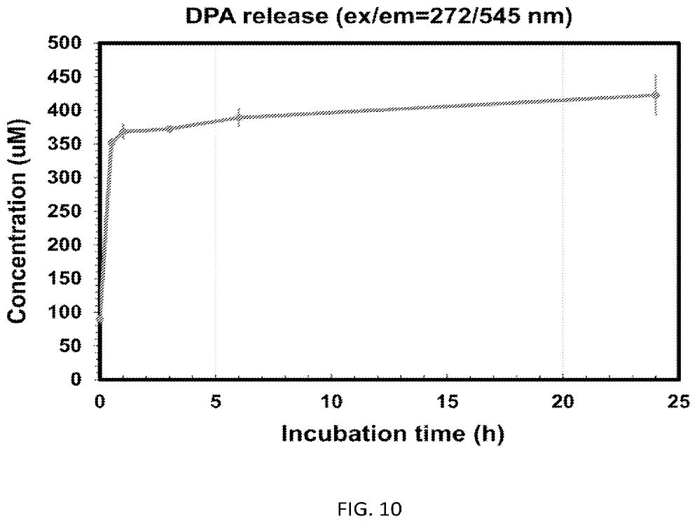

To further elucidate the calcium derived swelling effect on the microspheres, dipicolinic acid (DPA) that is a molecule accumulated in the core of spores in many endospore-forming species such as Bacillus subtilis and Clostridium novyi^{20, 40}. DPA accumulation in core of endospore supports the protection of genomic DNA and essential proteins from harsh environment such as heat⁴¹, UV⁴², and oxidative conditions⁴³. DPA is known as a metal chelating molecule, which can be used for preparation of transition metal complexes for ion chromatography⁴⁴. Also, approximately 10 wt % of dry mass of bacterial spores are filled with DPA, which is exceptionally high contents as single molecules⁴⁵. DPA highly loaded in C. novyi spores were used because DPA can form complex with many kinds of metal ions, and also the amount loaded in spores may be highly concentrated enough for the complete degradation of calcium alginate microspheres. For detection of DPA released out during germination process, terbium ions that have specific coordination with DPA in the ratio of 1 to 3 (Tb³⁺ to DPA) were utilized. Terbium ions coordinated with DPA molecules are highly fluorescent with a maximum excitation at 280 nm and an emission at 545 nm⁴⁶. Therefore, the specific fluorescence of Tb(DPA)₃ could help distinguishing the presence of DPA from other molecules in C. novyi media. The fluorescence was observed with the sample collected from predetermined time points up to 24 hours, and 1 mM of TbCl₃ solution was added to the samples (final concentration of Tb³⁺: 0.1 mM). As shown in FIG. 10, the release of DPA during germination was detected after 1 hour of incubation in hypoxic condition, and the maximum release of DPA was near 400 μM from 1×10⁷ spores in 1 ml of media. It can be a rational expectation that significant release of DPA that may chelate calcium ions in hydrogel matrix of calcium alginate microspheres could degrade the internal crosslinks and the morphology of calcium alginate microspheres.

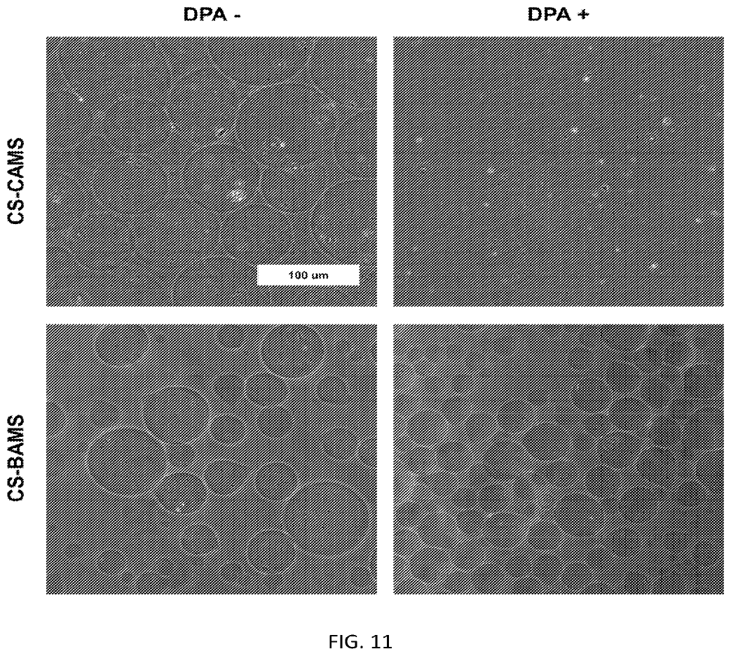

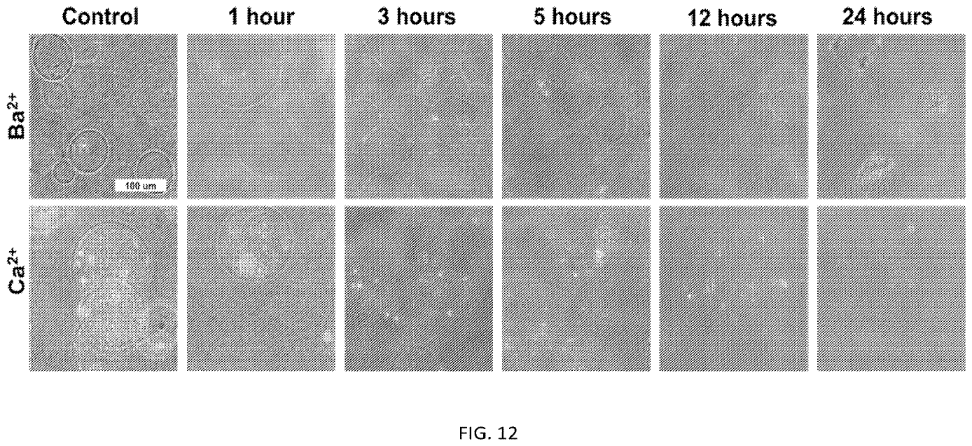

To demonstrate DPA effect on calcium alginate microspheres, a comparative experiment was designed with two types of alginate microspheres made of calcium (CS-CAMS) and barium ions (CS-BAMS) as ionic crosslinkers. Both of alginate microsphere were synthesized with same spraying-coagulation method. 30 mM of DPA solution was added into the sample deposited in 6-well cell-culture dish (final concentration of DPA: 400 uM). Surprisingly, after 1 minutes of incubation with DPA solution, the sample containing CS-CAMSs did not show any microspheres observed in optical microspheres, indicating that CS-CAMSs were completely degraded by treatment of DPA. The incorporated spores could be observed in microscopic image in FIG. 11. On the other hand, CS-BAMS made with barium ions did not show morphological disruption by the treatment of DPA. To further elucidate this phenomenon, CS-CAMSs and CS-BAMSs were incubated in hypoxic condition to trigger the germination of spores encapsulated in microspheres. Both of CS-CAMS and CS-BAMS could play a role of bacterial scaffolds to generate viable vegetative bacteria. Unexpectedly, CS-BAMS did not have any morphological defects supported by the DPA treatment assay (FIG. 12), and the bacteria germinated in CS-BAMSs were captured and immobilized inside of the microsphere matrix (24-hour post-incubation). These results strongly suggested that internally released DPA is the critical factor for the disruption of calcium alginate microspheres. Plus, the degradation phenomenon of calcium alginate by the germination of C. novyi could expand the potential of CS-CAMS as a bacterial delivery vehicle that can not only provide proper germination environment but also release living bacteria for attacking targeted region.

### Example 5 Anticancer Effect of CS-CAMS and Embolization

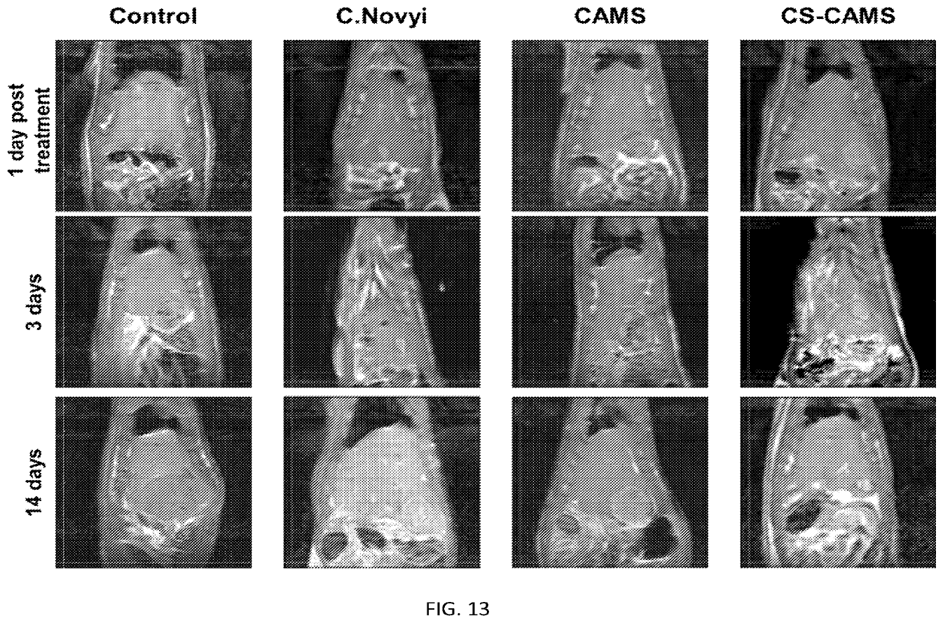

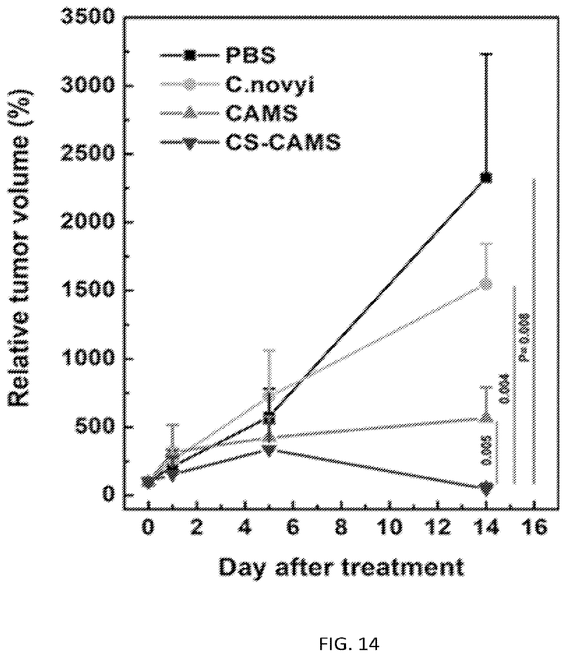

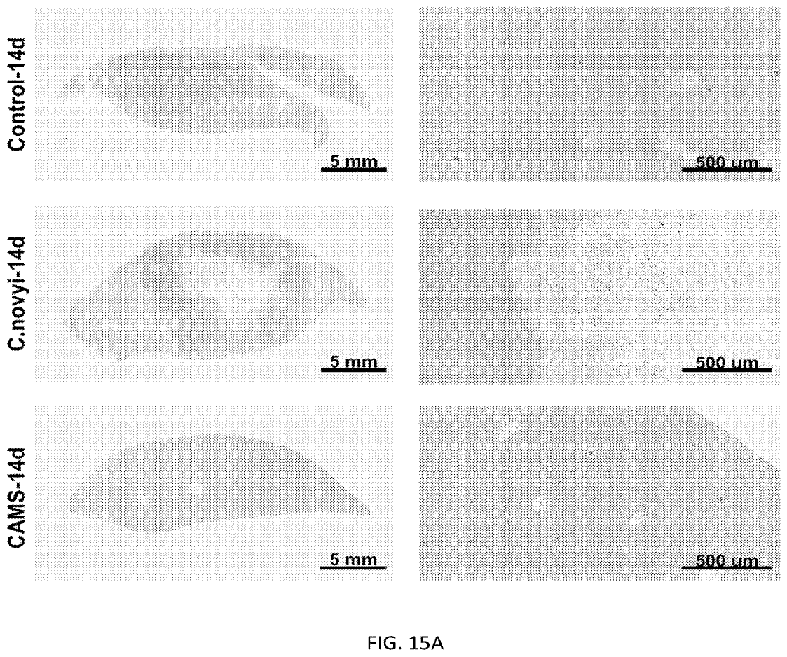

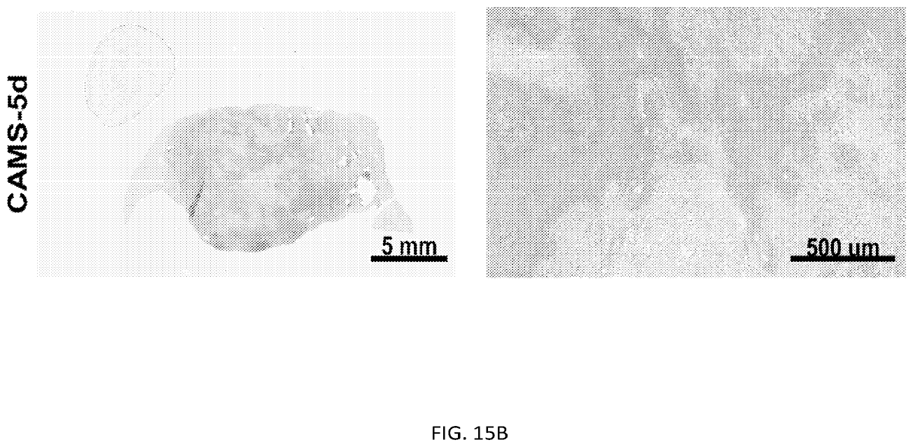

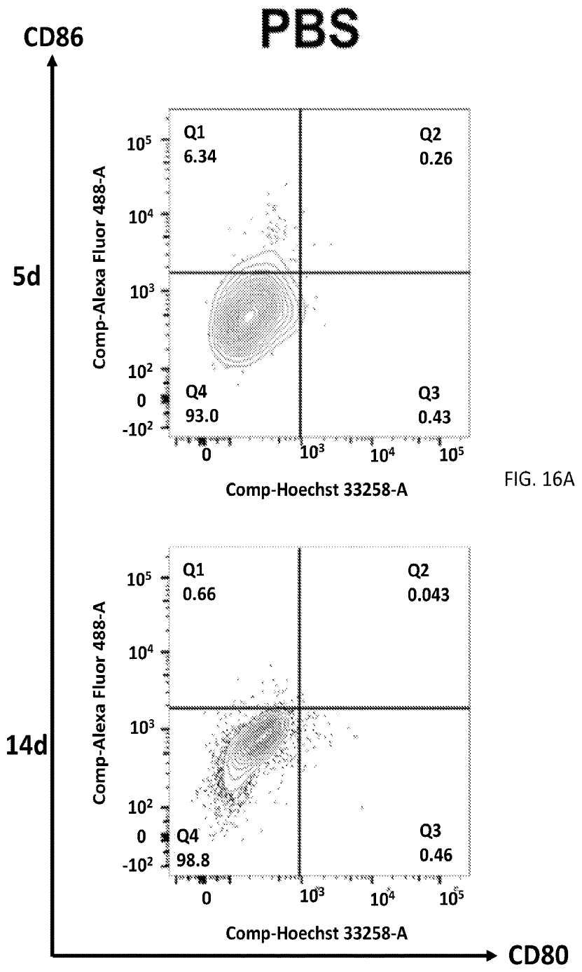

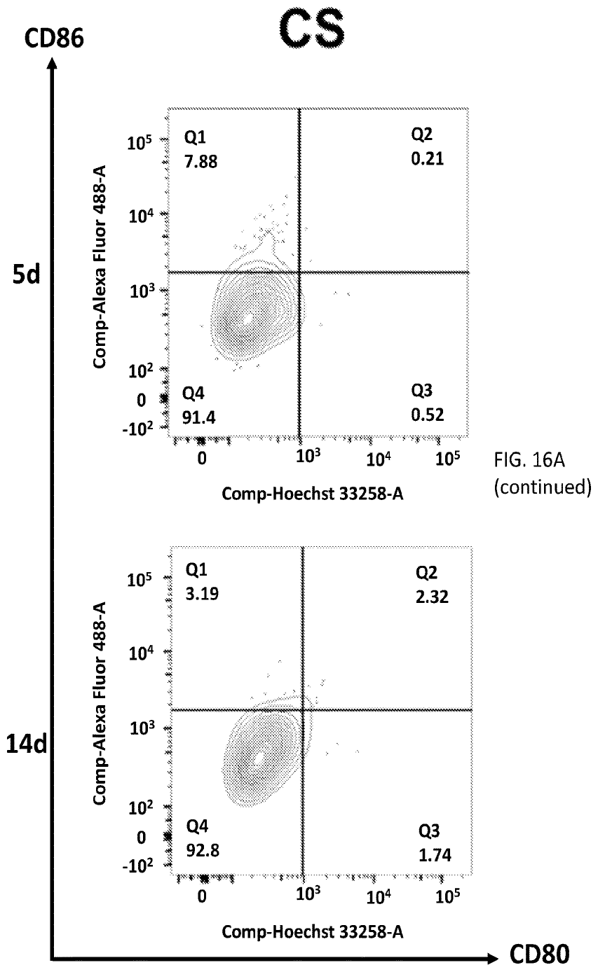

To determine the anti-tumor effect of CS-CAMSs, N1S1 cells, orthotropic hepatocellular carcinoma cells, were induced in the liver of Sprague Dawley rats by laparotomy and direct injection into the right lobe of the liver. After the confirmation of tumor lesion in preclinical MRI, the rats were treated with PBS as control, C. novyi, CAMSs, and CS-CAMS by using trans-arterial infusion technique. After the treatment of samples, the tumor growth was monitored by MRI (1-, 3-, and 14-days post-treatment), and the tumor size of the CS-CAMSs-treated group showed the greatest tumor inhibition compared to other groups (FIG. 13). Measurement of tumor volume shows the level of inhibition over the 14 days post treatment with the CS-CAMSs-treated group maintaining the greatest tumor inhibition compared to other groups (FIG. 14). This indicates the bacterial therapy combined with trans-arterial embolization therapy could act synergistically.

Histological sections of tumor treated with PBS, C. novyi and CS-CAMS after the 14-days post treatment were also prepared with gram staining for identifying the tumor response and the distribution of C. novyi. As shown in FIG. 15A, intraarterially treated C. novyi showed severe abscess that is a typical side effect due to the selective growth of C. novyi in necrotic region of the tumor. Also, the bacteria stained with purple color by crystal violet were mainly located in necrotic region, and there were not many bacteria visible in the tumor rim where the sufficient blood vessels supplied oxygen. On the other hand, tumor lesions could not be found in the histological sections in the group treated with CS-CAMS. To understand the effect of CS-CAMSs on N1S1 hepatoma in rats, the histological sections of the tumor treated with CS-CAMSs after the 5-days post treatment were prepared with gram staining (FIG. 15B). Surprisingly, strong purple color was shown in all over the tumor region, and the tumor rim with a plenty of vasculatures also showed the presence of purple pigment, indicating not only the bacterial distribution was significantly larger but also C. novyi bacteria could escape from the alginate microspheres and could invaded tumor rim through the vasculatures that could not provide oxygen due to induced hypoxia.

### Example 6 Immune Responses after Treatment of CS-CAMSs

To investigate immune modulation on tumor microenvironment by IA-infused CS-CAMSs, major immune cells known to be related to tumor lysis by C. novyi infection were analyzed by flow cytometry. Anti-cancer C. novyi therapy is also known as bacteria immunotherapy by strong immune boosting activity of bacterial infection and massive tumor lysis³.

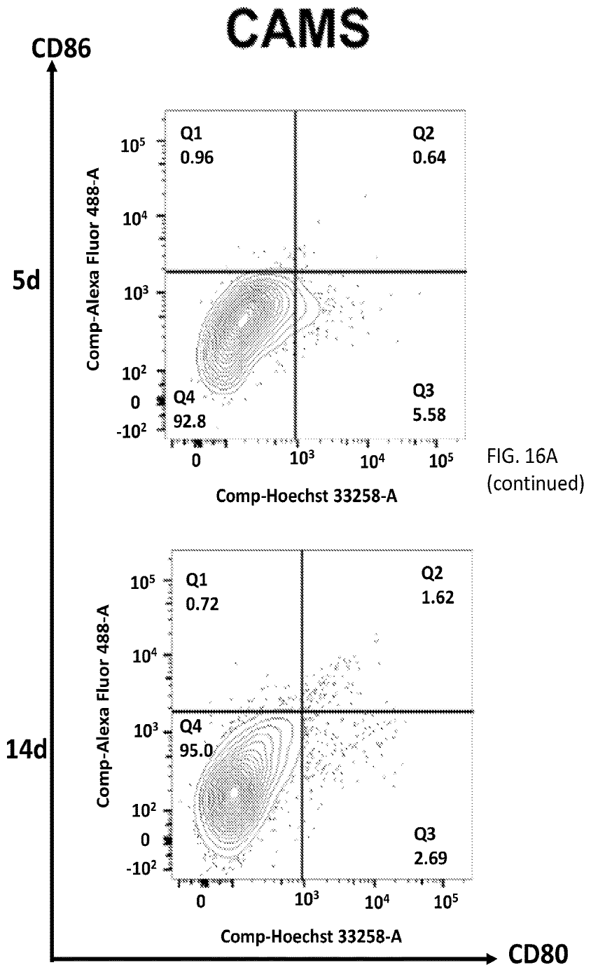

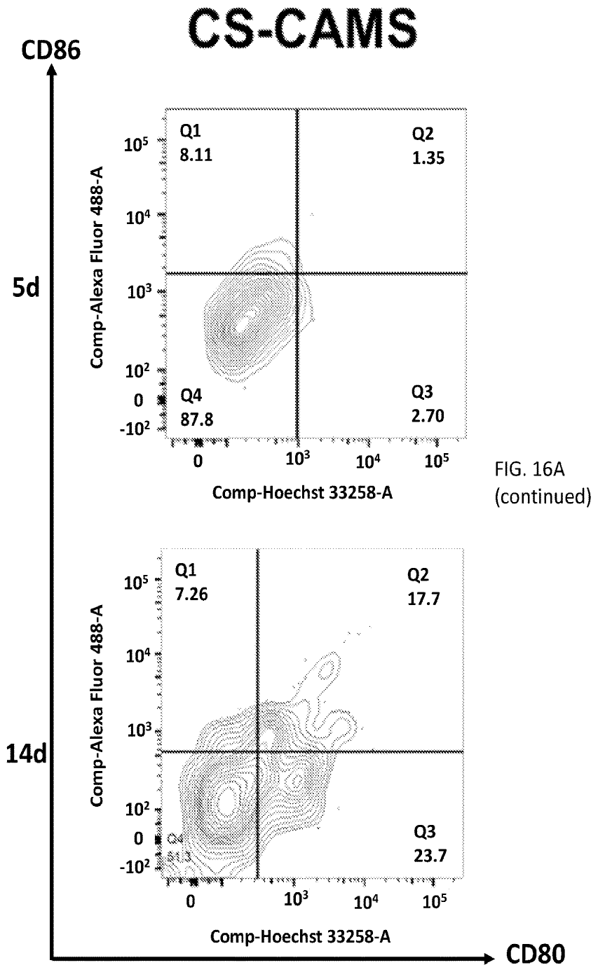

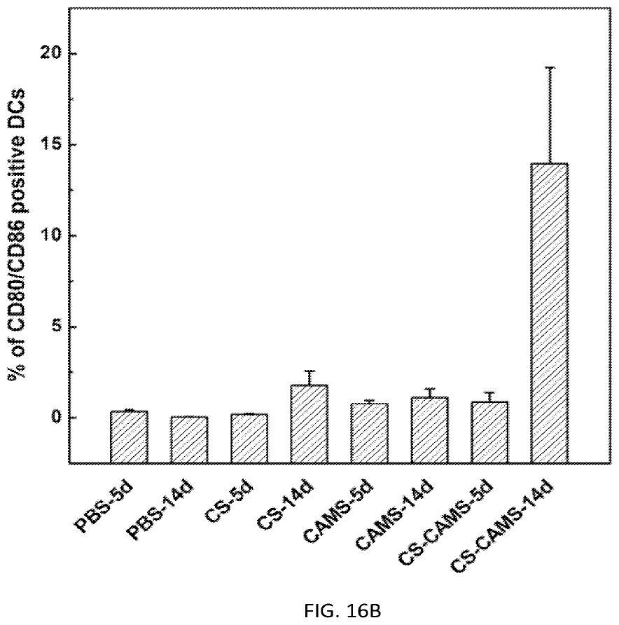

First, the population analysis of dendritic cells (DCs) in the tumor that are the major antigen presenting cells modulating adaptive immune system by activating or deactivating T cells⁴⁷ was conducted. As shown in FIGS. 16A-16B, immunogenic conversion of CS-CAMSs enhanced the maturation of dendritic cells in 14-day post treatment around 13.95±5.30%, showing elevation of CD80/CD86 that are representative costimulatory molecules expression⁴⁸. Comparably, the IA-infused C. novyi spores did not show significant increase of DC maturation (1.77±0.78%), indicating that the antigen supply and proinflammatory signal might not occur significantly at the rim and periphery of tumor where most readily activable DCs stay while the DCs inside the deeper tumor region are relatively inactivated due to tumor-suppressor factors^{49, 50}. This result indicated that intraarterial infusion of CS-CAMS might expand the area of tumor modulated with the proinflammatory responses by effective tumor cell death caused by bacterial infection.

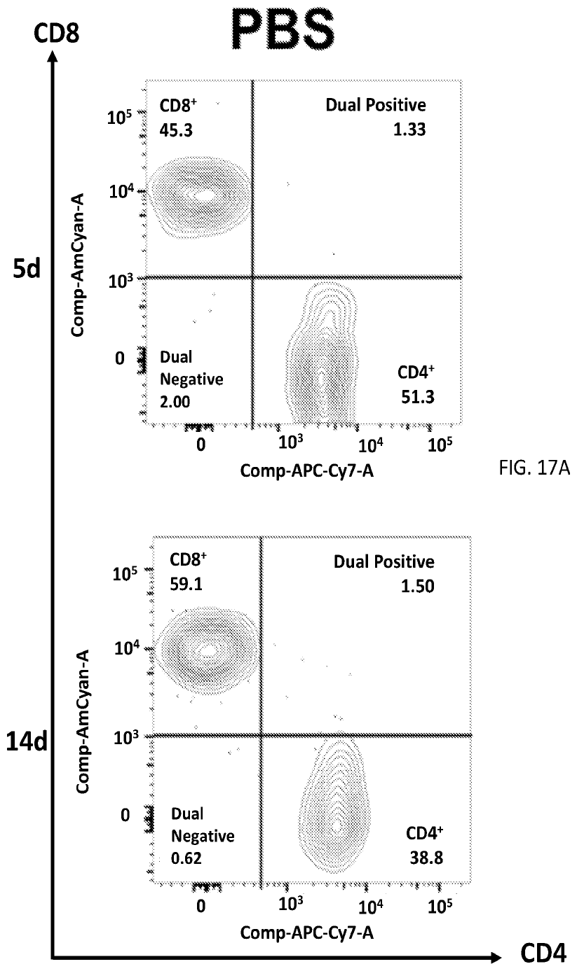

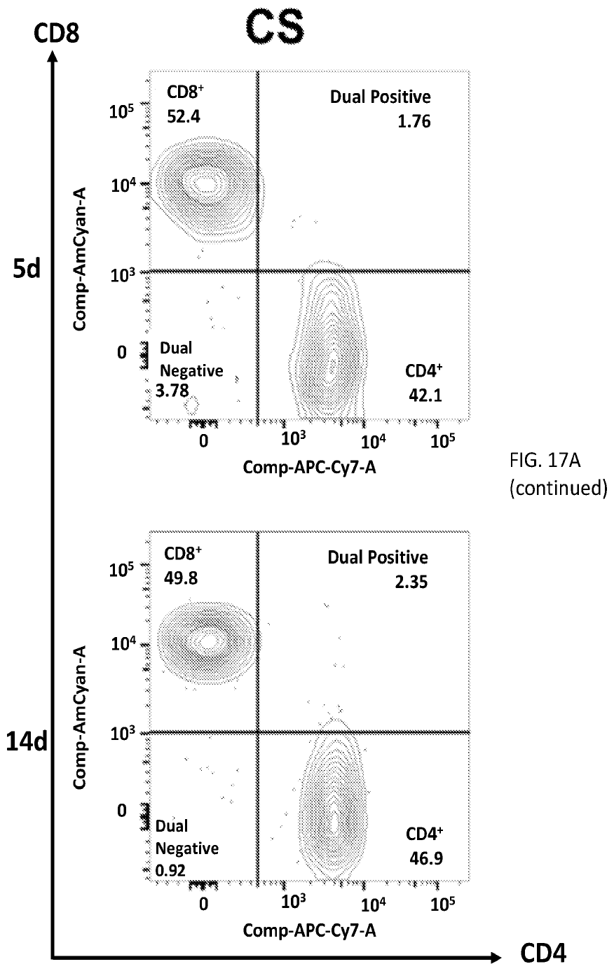

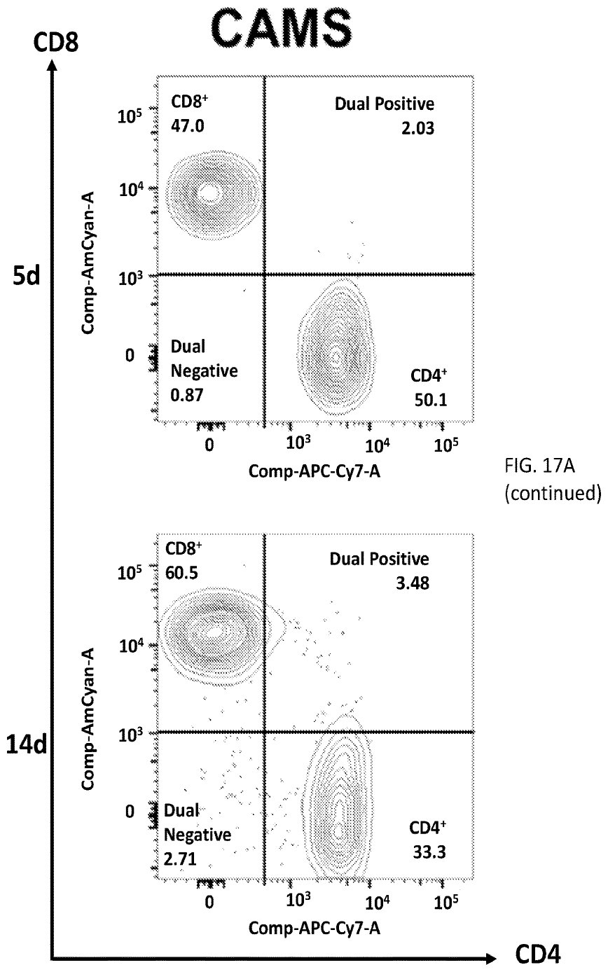

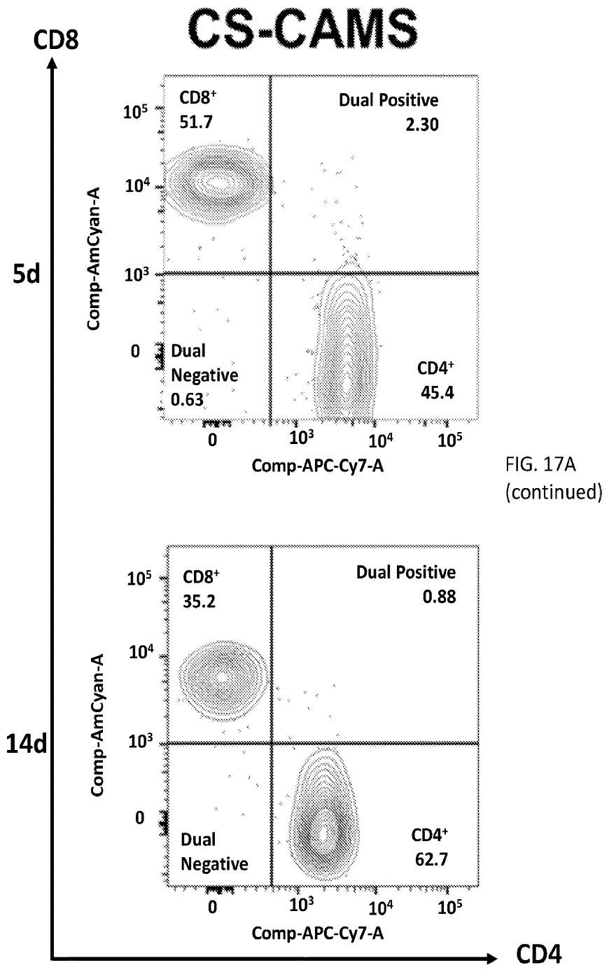

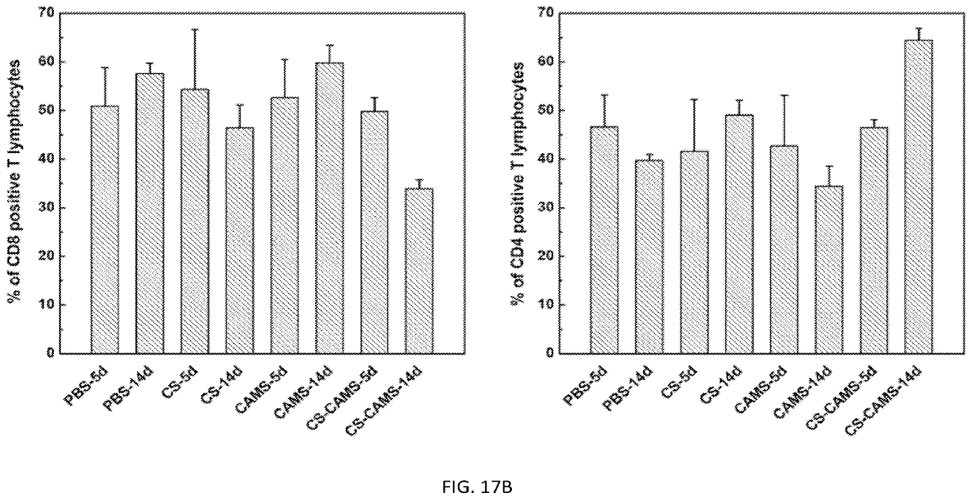

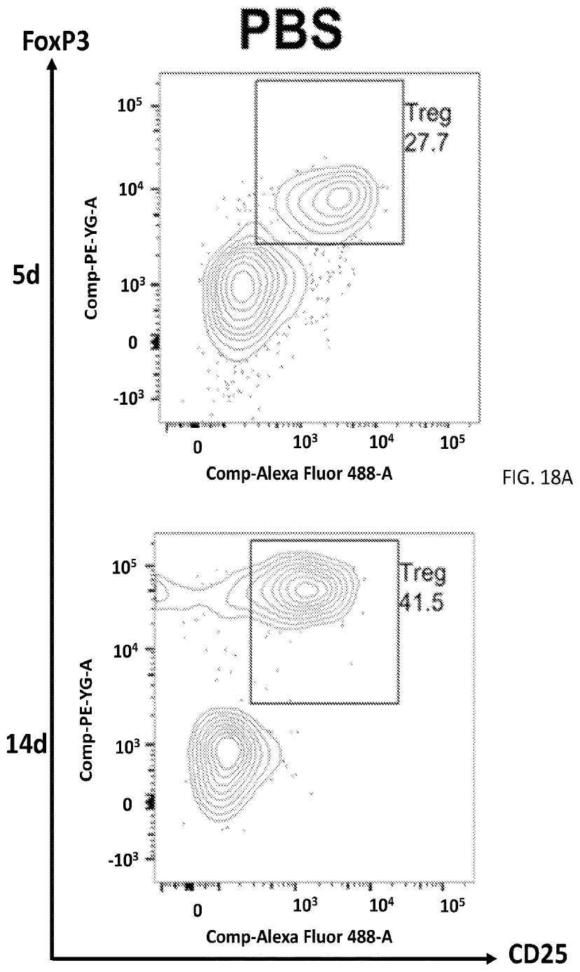

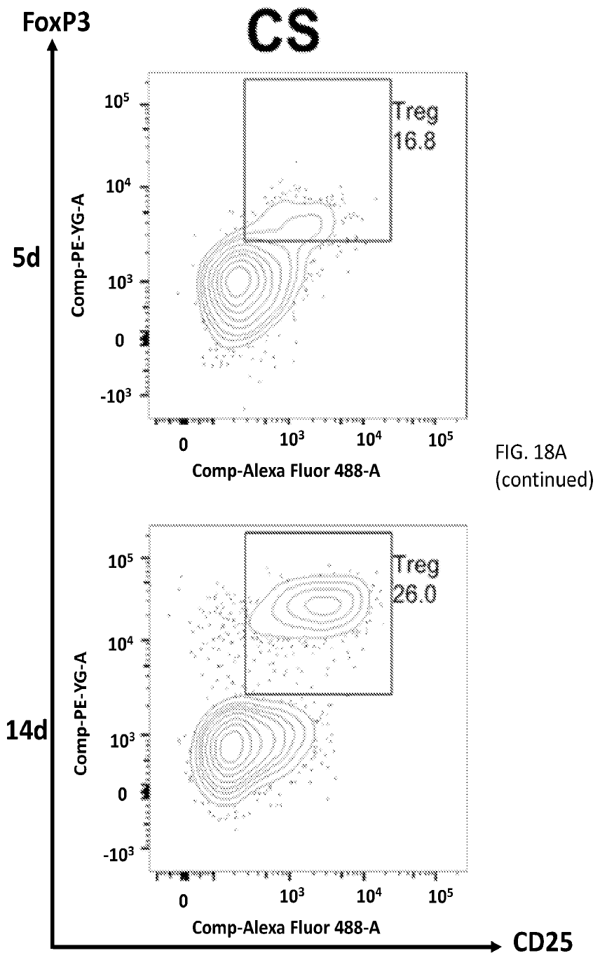

Next, to evaluate T lymphocyte infiltration into tumor, T cell subsets in tumor were analyzed with flow cytometric analysis. As shown in FIGS. 17A-17B, on 5-days post treatment, there was no significant difference in the CD8+ T cell population in the groups of PBS treated group (50.9±7.9%), C. novyi treated group (54.3±12.3%), CAMS treated group (52.6±7.9%), and CS-CAMS treated group (49.8±2.8%), and the ratio of CD4+ T cells were slightly reduced in C. novyi and CAMS treated group. On the other hand, unexpectedly, CS-CAMS treated group on 14-days post treatment showed significant increase of CD4+ population to 64.5±2.5%. The CD4+ T lymphocytes were increased in the 14-day post treatment of CS-CAMS approximately 39% compared to the data of 5-day post treatment. In the contrary, CAMS treated groups even showed reduced portion of CD 4+ T lymphocyte population, and C. novyi treated group showed no significant difference from the tumor after 5-day post treatment.

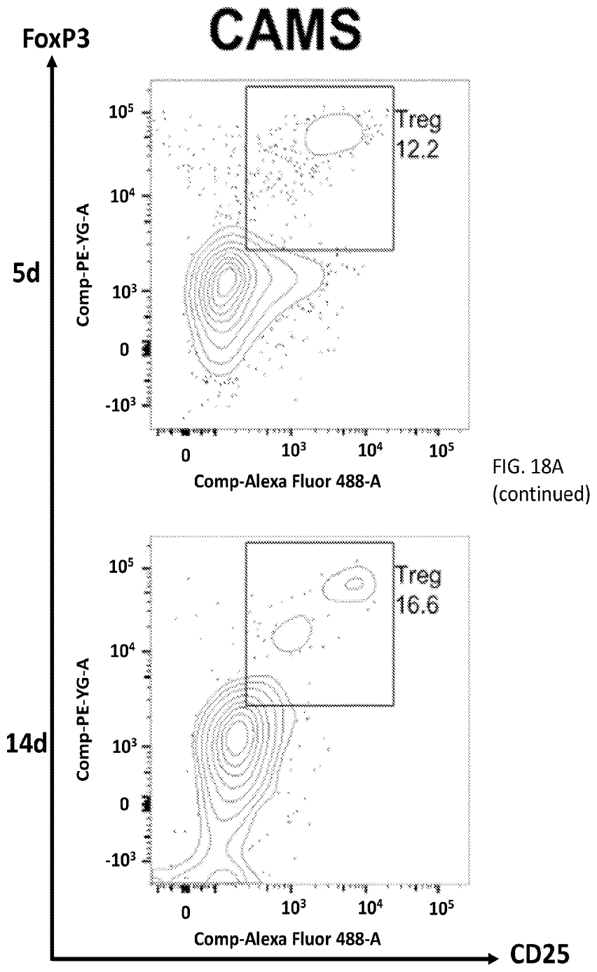

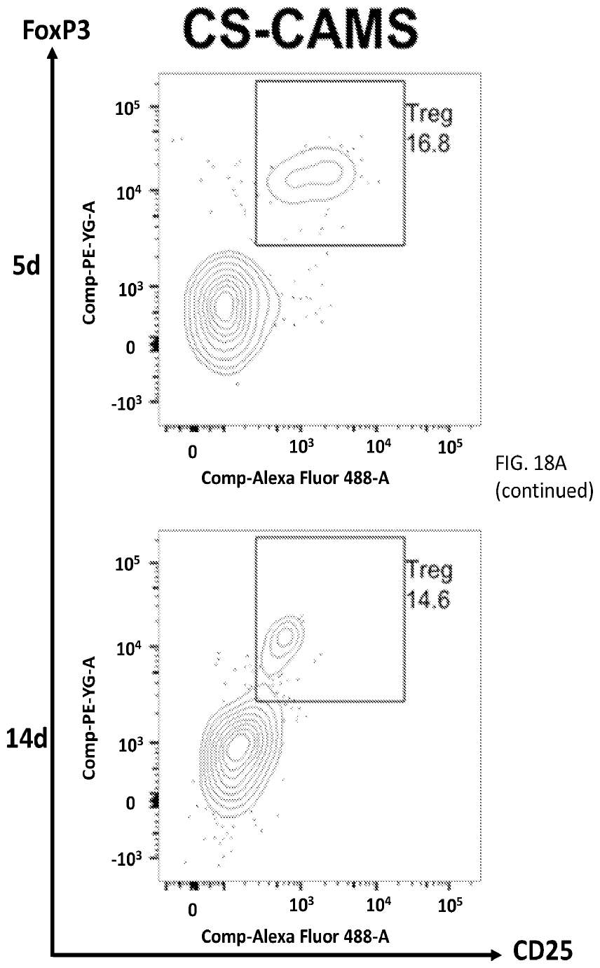

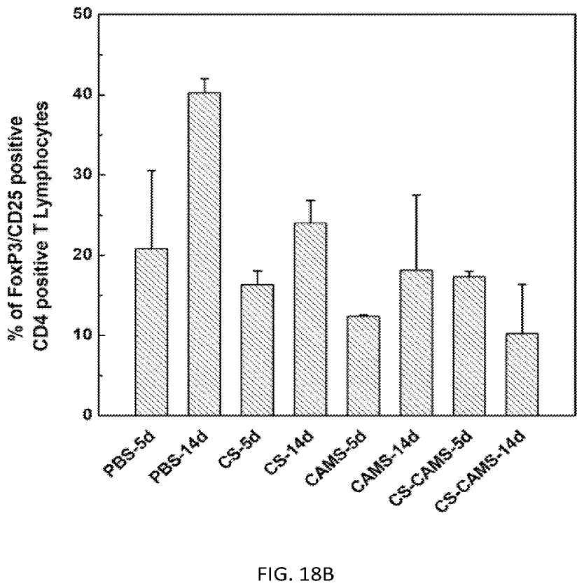

To further elucidate the information of CD4+ T cells elevated with treatment of CS-CAMSs, regulatory T cell population in CD4+ lymphocyte was measured. Surprisingly, regulatory T cell was not a contributor of increased population of the CD4+ lymphocyte in the previous analysis as shown in FIGS. 18A-18B. After 14-days post CS-CAMS treatment, regulatory T cell population was decreased approximately 41% compared from the population observed in 5-days post treatment, while all other groups (PBS, C. novyi spore, and CAMS) showed the increased regulatory T cell population while tumor was growing. Both individual treatments (C. novyi spore only and blank alginate microspheres) showed decreased number of regulatory T cells indicating the tumor microenvironment was less suppressive modulated by either C. novyi treatment or embolization.

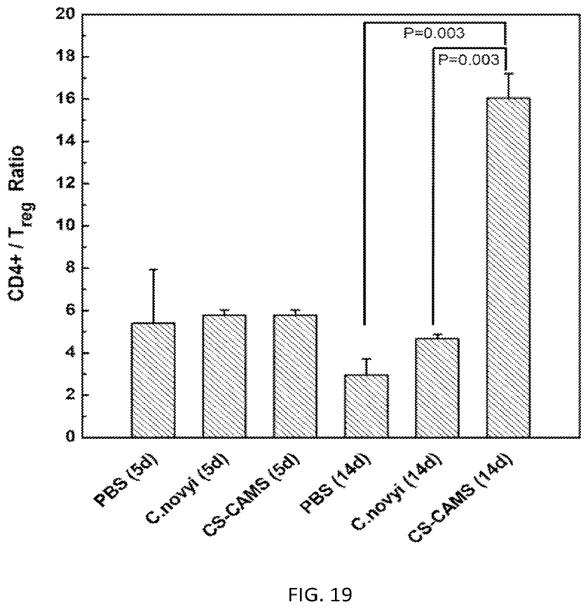

Additionally, results indicated the tumor response of CS-CAMSs can be dominantly mediated by CD4+ T cell subsets that do not include regulatory T cell. The analysis of regulatory T cell to CD4 ratio also supported the decreased number of regulatory T cell among CD4+ T cells, and this tendency was maximized with CS-CAMS treatment that provided bacterial infection and hypoxia (FIG. 19). On 14 days post treatment, CD4+/Treg value was 16.0±1.2, which was significantly higher than PBS treated group (2.9±0.8) and C. novyi spore treated group (4.7±0.2). Therefore, the significant reduction of regulatory T cell population by the combination of bacteria treatment and embolization could be correlated with the complete tumor response in most cases, providing increased maturation of DCs due to immunogenic bacteria and cytokines by enlarged area and suppressing immunosuppressive regulatory T cells.

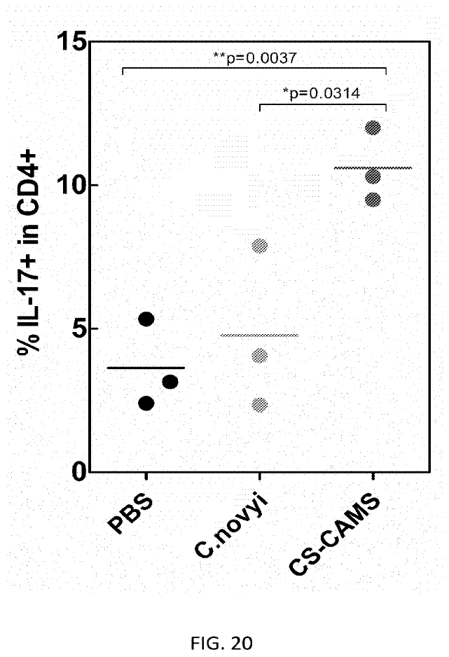

There are several reports supporting CD4+ T cell mediated immune response by the oncolytic bacterial therapies⁵¹. Also, regulatory T cells and T-helper 17 (Th17) cells has antagonistic polarization against each other by the physiological and pathological condition of tissue⁵². Interestingly, the condition that CS-CAMSs given to the tumor was favorable for the polarization toward Th17 subset. Th17 cells are known that they are predominantly differentiated from naïve CD4+ T cells by microbial and/or viral infection, and the elevation of Th17 (10.6% in CD4+ T cells) was observed by the treatment of CS-CAMS that was significantly higher compared to the intra-arterial treatment of C. novyi spores (4.8%) and the treatment of PBS as a control (3.6% in CD4+ T cells.) (FIG. 20).

Also, it is known that differentiation of naïve CD4+ T cells could be biased to Th17 in the presence of HIF-1α, which is expressed as a responder to hypoxia^{53, 54}. Plus, trans-arterial embolization can induce the infiltration of Th17 in the tumor⁵⁵. Also, FoxP3, the master transcription factor of regulatory T cells is known to be ubiquitinated and degraded in the presence of HIF-1α⁵⁴. Therefore, embolization can cause severe hypoxia on the tumor, and C. novyi released out from the alginate microspheres may enhance the activity of Th17 cells and decrease the function of regulatory T cells.

### Example 7 Tumor Response after Trans-Arterial Treatment with CS-CAMS

To assess the anti-tumor effect of CS-CAMSs, orthotropic hepatocellular carcinoma cells N1S1 were induced in the liver of Sprague Dawley rats by laparotomy and direct injection into the right lobe of the liver. After the confirmation of tumor lesion in preclinical MRI, the rats were treated with PBS as control, C. novyi-NT, blank alginate microspheres (CAMSs), and CS-CAMS by using trans-arterial infusion technique. After the treatment of samples, the tumor growth was monitored by MRI at 1, 3, and 14 days post-treatment. The tumor size of the CS-CAMSs-treated group showed the greatest tumor inhibition compared to other groups (FIG. 21). This indicates the bacterial therapy combined with trans-arterial embolization therapy can act synergistically.

In this description, it will be readily apparent to one skilled in the art that varying substitutions and modifications may be made to the invention disclosed herein without departing from the scope and spirit of the invention. The invention illustratively described suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of non-patent references are made throughout this disclosure. The cited references are incorporated by reference in their entireties. In the event that there is an inconsistency between a definition of a term in this specification as compared to a definition of the term in a cited reference, the term should be interpreted based on the definition in the specification.

### REFERENCES

1. Huang, X.; Pan, J.; Xu, F.; Shao, B.; Wang, Y.; Guo, X.; Zhou, S., Bacteria-based cancer immunotherapy. Advanced Science 2021, 8 (7), 2003572.
2. Zhou, S.; Gravekamp, C.; Bermudes, D.; Liu, K., Tumour-targeting bacteria engineered to fight cancer. Nature Reviews Cancer 2018, 18 (12), 727-743.
3. Janku, F.; Zhang, H. H.; Pezeshki, A.; Goel, S.; Murthy, R.; Wang-Gillam, A.; Shepard, D. R.; Helgason, T.; Masters, T.; Hong, D. S., Intratumoral Injection of Clostridium novyi-NT Spores in Patients with Treatment-refractory Advanced Solid TumorsPhase I Study of Intratumoral Clostridium novyi-NT. Clinical Cancer Research 2021, 27 (1), 96-106.
4. Diaz Jr, L. A.; Cheong, I.; Foss, C. A.; Zhang, X.; Peters, B. A.; Agrawal, N.; Bettegowda, C.; Karim, B.; Liu, G.; Khan, K., Pharmacologic and toxicologic evaluation of C. novyi-NT spores. Toxicological Sciences 2005, 88 (2), 562-575.
5. Desnous, C.; Guillaume, D.; Clivio, P., Spore photoproduct: a key to bacterial eternal life. Chemical reviews 2010, 110 (3), 1213-1232.
6. Knecht, L. D.; Pasini, P.; Daunert, S., Bacterial spores as platforms for bioanalytical and biomedical applications. Analytical and bioanalytical chemistry 2011, 400 (4), 977-989.
7. Heppner, F.; Möse, J., The liquefaction (oncolysis) of malignant gliomas by a non pathogenic Clostridium. Acta Neurochirurgica 1978, 42 (1), 123-125.
8. Roberts, N. J.; Zhang, L.; Janku, F.; Collins, A.; Bai, R.-Y.; Staedtke, V.; Rusk, A. W.; Tung, D.; Miller, M.; Roix, J., Intratumoral injection of Clostridium novyi-NT spores induces antitumor responses. Science translational medicine 2014, 6 (249), 249ra111-249ra111.
9. Chabner, B. A.; Roberts, T. G., Chemotherapy and the war on cancer. Nature Reviews Cancer 2005, 5 (1), 65-72.
10. Romond, E. H.; Perez, E. A.; Bryant, J.; Suman, V. J.; Geyer Jr, C. E.; Davidson, N. E.; Tan-Chiu, E.; Martino, S.; Paik, S.; Kaufman, P. A., Trastuzumab plus adjuvant chemotherapy for operable HER2-positive breast cancer. New England journal of medicine 2005, 353 (16), 1673-1684.
11. Garon, E. B.; Rizvi, N. A.; Hui, R.; Leighl, N.; Balmanoukian, A. S.; Eder, J. P.; Patnaik, A., Aggarwal, C.; Gubens, M.; Horn, L., Pembrolizumab for the treatment of non-small-cell lung cancer. New England Journal of Medicine 2015, 372 (21), 2018-2028.
12. Small, E. J.; Tchekmedyian, N. S.; Rini, B. I.; Fong, L.; Lowy, I.; Allison, J. P., A pilot trial of CTLA-4 blockade with human anti-CTLA-4 in patients with hormone-refractory prostate cancer. Clinical Cancer Research 2007, 13 (6), 1810-1815.
13. Brahmer, J. R., Tykodi, S. S.; Chow, L. Q.; Hwu, W.-J.; Topalian, S. L., Hwu, P.; Drake, C. G., Camacho, L. H.; Kauh, J.; Odunsi, K., Safety and activity of anti-PD-L1 antibody in patients with advanced cancer. New England Journal of Medicine 2012, 366 (26), 2455-2465.
14. Galluzzi, L.; Buqué, A.; Kepp, O.; Zitvogel, L.; Kroemer, G., Immunogenic cell death in cancer and infectious disease. Nature Reviews Immunology 2017, 17 (2), 97-111.
15. Kasinskas, R. W.; Forbes, N. S., Salmonella typhimurium lacking ribose chemoreceptors localize in tumor quiescence and induce apoptosis. Cancer research 2007, 67 (7), 3201-3209.
16. Beutler, B.; Rietschel, E. T., Innate immune sensing and its roots: the story of endotoxin. Nature Reviews Immunology 2003, 3 (2), 169-176.
17. Skountzou, I.; del Pilar Martin, M.; Wang, B.; Ye, L.; Koutsonanos, D.; Weldon, W.; Jacob, J.; Compans, R. W., Salmonella flagellins are potent adjuvants for intranasally administered whole inactivated influenza vaccine. Vaccine 2010, 28 (24), 4103-4112.
18. Chandra, D.; Jahangir, A., Quispe-Tintaya, W.; Einstein, M.; Gravekamp, C., Myeloid-derived suppressor cells have a central role in attenuated Listeria monocytogenes-based immunotherapy against metastatic breast cancer in young and old mice. British journal of cancer 2013, 108 (11), 2281-2290.
19. MacLennan, J. D., The histotoxic clostridial infections of man. Bacteriological reviews 1962, 26 (2_pt_1-2), 177-274.
20. Olguín-Araneda, V.; Banawas, S.; Sarker, M. R.; Paredes-Sabja, D., Recent advances in germination of Clostridium spores. Research in microbiology 2015, 166 (4), 236-243.
21. Dang, L. H.; Bettegowda, C.; Huso, D. L.; Kinzler, K. W.; Vogelstein, B., Combination bacteriolytic therapy for the treatment of experimental tumors. Proceedings of the National Academy of Sciences 2001, 98 (26), 15155-15160.
22. Bettegowda, C.; Huang, X.; Lin, J., Cheong, I.; Kohli, M.; Szabo, S. A.; Zhang, X.; Diaz, L. A.; Velculescu, V. E.; Parmigiani, G., The genome and transcriptomes of the anti-tumor agent Clostridium novyi-NT. Nature biotechnology 2006, 24 (12), 1573-1580.
23. Agrawal, N.; Bettegowda, C.; Cheong, I.; Geschwind, J.-F.; Drake, C. G.; Hipkiss, E. L.; Tatsumi, M.; Dang, L. H.; Diaz Jr, L. A.; Pomper, M., Bacteriolytic therapy can generate a potent immune response against experimental tumors. Proceedings of the National Academy of Sciences 2004, 101 (42), 15172-15177.
24. Dailey, K. M.; Jacobson, R. I.; Johnson, P. R.; Woolery, T. J.; Kim, J.; Jansen, R. J.; Mallik, S.; Brooks, A. E., Methods and Techniques to Facilitate the Development of Clostridium novyi NT as an Effective, Therapeutic Oncolytic Bacteria. Frontiers in Microbiology 2021, 12, 624618.
25. Cheong, I.; Huang, X.; Bettegowda, C.; Diaz Jr, L. A.; Kinzler, K. W.; Zhou, S.; Vogelstein, B., A bacterial protein enhances the release and efficacy of liposomal cancer drugs. Science 2006, 314 (5803), 1308-1311.
26. Virmani, S.; Rhee, T. K.; Ryu, R. K.; Sato, K. T.; Lewandowski, R. J.; Mulcahy, M. F.; Kulik, L. M.; Szolc-Kowalska, B.; Woloschak, G. E.; Yang, G.-Y. J. J. o. V.; Radiology, I., Comparison of hypoxia-inducible factor-1α expression before and after transcatheter arterial embolization in rabbit VX2 liver tumors. 2008, 19 (10), 1483-1489.
27. Chen, C.; Tsang, Y.-M.; Hsueh, P.-R.; Huang, G.-T.; Yang, P.-M.; Sheu, J.-C.; Lai, M.-Y.; Chen, P.-J.; Chen, D.-S., Bacterial infections associated with hepatic arteriography and transarterial embolization for hepatocellular carcinoma: a prospective study. Clinical infectious diseases 1999, 29 (1), 161-166.
28. Aungst, M.; Wilson, M.; Vournas, K.; McCarthy, S., Necrotic leiomyoma and gram-negative sepsis eight weeks after uterine artery embolization. Obstetrics & Gynecology 2004, 104 (5), 1161-1164.
29. Kochan, T. J.; Somers, M. J.; Kaiser, A. M.; Shoshiev, M. S.; Hagan, A. K.; Hastie, J. L.; Giordano, N. P.; Smith, A. D.; Schubert, A. M.; Carlson Jr, P. E., Intestinal calcium and bile salts facilitate germination of Clostridium difficile spores. PLoS pathogens 2017, 13 (7), e1006443.
30. Li, R. H.; Altreuter, D. H.; Gentile, F. T., Transport characterization of hydrogel matrices for cell encapsulation. Biotechnology and bioengineering 1996, 50 (4), 365-373.
31. Donnelly, M. L.; Fimlaid, K. A.; Shen, A., Characterization of Clostridium difficile spores lacking either SpoVAC or dipicolinic acid synthetase. Journal of bacteriology 2016, 198 (11), 1694-1707.
32. Jenkins, A. L.; Murray, G. M., Enhanced luminescence of lanthanides: determination of europium by enhanced luminescence. Journal of chemical education 1998, 75 (2), 227.
33. Li, X.; Liu, T.; Song, K.; Yao, L.; Ge, D.; Bao, C.; Ma, X.; Cui, Z., Culture of neural stem cells in calcium alginate beads. Biotechnology progress 2006, 22 (6), 1683-1689.
34. Moshaverinia, A.; Ansari, S.; Chen, C.; Xu, X.; Akiyama, K.; Snead, M. L.; Zadeh, H. H.; Shi, S., Co-encapsulation of anti-BMP2 monoclonal antibody and mesenchymal stem cells in alginate microspheres for bone tissue engineering. Biomaterials 2013, 34 (28), 6572-6579.
35. Pawar, S. N.; Edgar, K. J., Alginate derivatization: A review of chemistry, properties and applications. Biomaterials 2012, 33 (11), 3279-3305.
36. Riemann, H.; Ordal, Z. J., Germination of bacterial endospores with calcium and dipicolinic acid. Science 1961, 133 (3465), 1703-1704.
37. Nakamura, S.; Kimura, I.; Yamakawa, K.; Nishida, S., Taxonomic relationships among Clostridium novyi types A and B, Clostridium haemolyticum and Clostridium botulinum type C. Microbiology 1983, 129 (5), 1473-1479.
38. Prescott, J. F.; MacInnes, J. I.; Wu, A. K., Taxonomic relationships among the clostridia. Clostridial Diseases of Animals. Ames, IA: Wiley-Blackwell 2016, 2016, 3-5.
39. Sorg, J. A.; Sonenshein, A. L., Bile salts and glycine as cogerminants for Clostridium difficile spores. Journal of bacteriology 2008, 190 (7), 2505-2512.
40. Aronson, A. I.; Henderson, E.; Tincher, A., Participation of the lysine pathway in dipicolinic acid synthesis in Bacilluscereus T. Biochemical and Biophysical Research Communications 1967, 26 (4), 454-460.
41. Church, B. D.; Halvorson, H., Dependence of the heat resistance of bacterial endospores on their dipicolinic acid content. Nature 1959, 183 (4654), 124-125.
42. Slieman, T. A.; Nicholson, W. L., Role of dipicolinic acid in survival of Bacillus subtilis spores exposed to artificial and solar UV radiation. Applied and environmental microbiology 2001, 67 (3), 1274-1279.
43. Setlow, B.; Atluri, S.; Kitchel, R.; Koziol-Dube, K.; Setlow, P., Role of dipicolinic acid in resistance and stability of spores of Bacillus subtilis with or without DNA-protective α/β-type small acid-soluble proteins. Journal of bacteriology 2006, 188 (11), 3740-3747.
44. Cowan, J.; Shaw, M. J.; Achterberg, E. P.; Jones, P.; Nesterenko, P., The ion chromatographic separation of high valence metal cations using a neutral polystyrene resin dynamically modified with dipicolinic acid. Analyst 2000, 125 (12), 2157-2159.
45. Warth, A. D., Determination of dipicolinic acid in bacterial spores by derivative spectroscopy. Analytical biochemistry 1983, 130 (2), 502-505.
46. Barela, T. D.; Sherry, A. D., A simple, one-step fluorometric method for determination of nanomolar concentrations of terbium. Analytical biochemistry 1976, 71 (2), 351-352.
47. Guermonprez, P.; Valladeau, J.; Zitvogel, L., Théry, C., Amigorena, S., Antigen presentation and T cell stimulation by dendritic cells. Annual review of immunology 2002, 20 (1), 621-667.
48. Orabona, C.; Grohmann, U.; Belladonna, M. L.; Fallarino, F.; Vacca, C.; Bianchi, R.; Bozza, S.; Volpi, C.; Salomon, B. L.; Fioretti, M. C., CD28 induces immunostimulatory signals in dendritic cells via CD80 and CD86. Nature immunology 2004, 5 (11), 1134-1142.
49. Zou, W., Regulatory T cells, tumour immunity and immunotherapy. Nature Reviews Immunology 2006, 6 (4), 295-307.
50. Bennaceur, K.; Chapman, J.; Brikci-Nigassa, L.; Sanhadji, K.; Touraine, J.-l.; Portoukalian, J., Dendritic cells dysfunction in tumour environment. Cancer letters 2008, 272 (2), 186-196.
51. Wang, W.; Xu, H.; Ye, Q.; Tao, F.; Wheeldon, I.; Yuan, A.; Hu, Y.; Wu, J., Systemic immune responses to irradiated tumours via the transport of antigens to the tumour periphery by injected flagellate bacteria. Nature Biomedical Engineering 2022, 6 (1), 44-53.
52. Kimura, A.; Kishimoto, T., IL-6: regulator of Treg/Th17 balance. European journal of immunology 2010, 40 (7), 1830-1835.
53. Shi, L. Z.; Wang, R.; Huang, G.; Vogel, P.; Neale, G.; Green, D. R.; Chi, H., HIF1α-dependent glycolytic pathway orchestrates a metabolic checkpoint for the differentiation of TH17 and Treg cells. Journal of Experimental Medicine 2011, 208 (7), 1367-1376.
54. Dang, E. V.; Barbi, J.; Yang, H.-Y.; Jinasena, D.; Yu, H.; Zheng, Y.; Bordman, Z.; Fu, J.; Kim, Y.; Yen, H.-R., Control of TH17/Treg balance by hypoxia-inducible factor 1. Cell 2011, 146 (5), 772-784.
55. Avritscher, R.; Jo, N.; Polak, U.; Cortes, A. C.; Nishiofuku, H.; Odisio, B. C.; Takaki, H.; Tam, A. L.; Melancon, M. P.; Yevich, S., Hepatic arterial bland embolization increases Th17 cell infiltration in a syngeneic rat model of hepatocellular carcinoma. CardioVascular and Interventional Radiology 2020, 43 (2), 311-321.

1. An anaerobic oncolytic bacterial composition comprising anaerobic bacterium spores and calcium alginate microspheres.

2. The composition of claim 1, wherein the anaerobic bacterium spores are encapsulated in the calcium alginate microspheres.

3. The composition of claim 1, wherein the anaerobic bacterium spores can germinate in the calcium alginate microspheres.

4. The composition of claim 1, wherein the anaerobic bacterium spores can germinate in the microspheres under hypoxic conditions.

5. (canceled)

6. The composition of claim 1, wherein the anaerobic oncolytic bacterium spores are selected from species of Clostridium, Listeria, and Salmonella.

7. The composition of claim 6, wherein the anaerobic bacterium spores are nontoxic.

8. A method of delivering an anaerobic oncolytic bacterium to a tumor comprising delivering the composition of claim 1 to the tumor.

9. The method of claim 8, wherein the anaerobic bacterium spores germinate in the microspheres under hypoxic conditions.

10. (canceled)

11. The method of claim 8, wherein the composition is delivered by embolization.

12. (canceled)

13. The method of claim 11, wherein the embolization induces a hypoxic environment in the tumor.

14. The method of claim 13, wherein the anaerobic bacterium spores germinate in the microspheres.

15. The method of claim 14, wherein the microspheres degrade and release vegetative anaerobic oncolytic bacteria in the tumor.

16-17. (canceled)

18. The method of claim 14, wherein the anaerobic bacterium spores are nontoxic Clostridium novyi (C. novyi-NT) spores.

19. The method of claim 14, wherein C. novyi-NT spores use calcium ions in the microspheres for germination.

20-26. (canceled)

27. A method of treating a tumor in a subject comprising delivering the composition of claim 1 to the subject, wherein an anti-tumor response is induced in the tumor.

28-29. (canceled)

30. The method of claim 27, wherein the composition is delivered by embolization.

31. (canceled)

32. The method of claim 30, wherein the embolization induces a hypoxic environment in the tumor.

33. (canceled)

34. The method of claim 27, wherein the anaerobic bacterium spores are nontoxic Clostridium novyi (C. novyi-NT) spores.

35. The method of claim 34, wherein C. novyi-NT spores use calcium ions in the microspheres for germination.

36. (canceled)

37. The method of claim 35, wherein the microspheres degrade and release vegetative Clostridium novyi in the tumor.

38-44. (canceled)
